(19) United States
(12) Patent Application Publication
(10) Pub. No.: US 2012/0321665 A1
(43) Pub. Date: Dec. 20, 2012

(54) COMPOSITIONS AND METHODS FOR TREATING AIRWAY INFLAMMATORY DISEASES

(75) Inventors: Paul Bollyky, Seattle, WA (US); Gerald T. Nepom, Lopez, WA (US); Michael G. Kinsella, Seattle, WA (US)

(73) Assignee: BENAROYA RESEARCH INSTITUTE AT VIRGINIA MASON, Seattle, WA (US)

(21) Appl. No.: 13/523,736

(22) Filed: Jun. 14, 2012

Related U.S. Application Data
(63) Continuation-in-part of application No. PCT/US2010/060323, filed on Dec. 14, 2010.
(60) Provisional application No. 61/496,956, filed on Jun. 14, 2011.
(60) Provisional application No. 61/286,315, filed on Dec. 14, 2009.

(51) Int. Cl. A61K 39/35; A61P 11/06; A61K 39/36; A61P 11/08; A61P 11/04; A61P 11/00; A61P 37/08

(52) U.S. Cl. 424/275.1

(57) ABSTRACT

A composition for treating an airway inflammatory disease having high molecular weight, crosslinked hyaluronan and at least one aeroallergen is provided. A method of inducing immune tolerance to one or more aeroallergens in a mammalian subject suffering from or at risk of developing an airway inflammatory disease, and a method of treating a human subject suffering from or at risk of developing an airway inflammatory disease or condition of the lungs are also provided.

### CROSS-REFERENCES TO RELATED APPLICATIONS

This application claims the benefit of U.S. Provisional Application Ser. No. 61/496,956, filed on Jun. 14, 2011, and is a Continuation in Part of PCT/US2010/060323, filed Dec. 14, 2010, which claims the benefit of U.S. Provisional Application Ser. No. 61/286,315, filed Dec. 14, 2009, which are incorporated herein by reference.

### STATEMENT OF GOVERNMENT LICENSE RIGHTS

This invention was made with U.S. Government support under Grant Nos. 1K08DK080178-01 and R24 HL64387-06A1 awarded by the National Institutes of Health, and Grant No. W81XWH-07-01-0246 awarded by the U.S. Army Medical Research and Materiel Command. The U.S. Government has certain rights in this invention.

### FIELD OF THE INVENTION

The present invention relates to immunomodulatory hydrogel compositions and methods of using the compositions to deliver a set of agents to modulate an immune response. The invention also relates to compositions and methods for treating airway inflammatory diseases.

### BACKGROUND

Immune tolerance is central to the immune system's ability to differentiate between self and foreign proteins. Central tolerance is initially achieved during thymic selection by the deletion of self-reactive T cells. However, central tolerance is incomplete, and further immune regulation is required in the periphery. Peripheral mechanisms of T cell regulation include the induction of energy, activation induced cell death, and regulatory T cells.

In healthy individuals immune tolerance is established and maintained by populations of regulatory T cells. Within the CD4+ T lymphocyte cell population, three categories of regulatory T cells have been described: TH3 cells, Type 1 regulatory (“TR1”) cells (CD4+FoxP3−), and CD4+CD25+FoxP3+ T regulatory cells (“Treg”). TH3 cells function via the secretion of TGF-beta and can be generated in vitro by stimulation in the presence of IL-4 or in vivo through oral administration of low dose antigens (Chen et al., Science 265:1237-1240, 1994; Inobe et al., Eur. J. Immunol. 28:2780-2790, 1998).

Type 1 regulatory T cells (“TR1”) are CD4+FoxP3− cells that mediate immune tolerance to self and foreign antigens through the production of IL-10 and TGF-beta and are derived by stimulation of memory T cells in the presence of IL-10 (Groux et al., Nature 389:737-742, 1997; Groux et al., J. Exp. Med. 184:19-29, 1996). The immunosuppressive effects of IL-10, and thereby TR1, are mediated in large part via suppression of macrophage/monocyte functions, including expression of Class II MHC and costimulatory molecules such as IL-12 and CD80/CD86 (Moore, K., et al., Annu. Rev. Immunol. 19:683-765, 2001). Disorders in IL-10 production or signaling result in autoimmune diseases, particularly in the gastrointestinal tract, in both mice (Asseman, C., et al., J. Exp. Med. 190:995-1004, 1999) and humans (Glocker, E., et al., N. Engl. J. Med. 361:2033-2045, 2009). TR1 cells are derived from conventional T cells in peripheral tissues (Roncarolo, M., et al., Immunol. Rev. 212:28-50, 2006), however the factors which induce TR1 in vivo are unknown. TR1 have been generated in vitro by priming T cells with low-dose antigen in the presence of IL-10 (Groux et al., Nature 389:737-742, 1997), steroids and vitamin D (Pedersen, A., et al., Immunol. Letter 91:63-69, 2004), or anti-CD46 antibodies (Kemper, C., et al., Nature 421:388-392, 2003).

CD4+CD25+FoxP3+ regulatory T cells (“Treg”) are thought to function as a regulator of autoimmunity by suppressing the proliferation and/or cytokine production of CD4+CD25− T cell responder cells at the site of inflammation. Treg are a specialized subpopulation of CD4+ T cells that maintain immune homeostasis in a variety of contexts (Sakaguchi, S., et al., Curr. Top. Microbiol. Immunol. 305:51-66, 2006). The suppressive capacity of Treg correlates with expression levels of the transcription factor FoxP3 (Sakaguchi, S., et al., 2006, supra). Treg mediate immune suppression by multiple mechanisms, including production of IL-10, a critical anti-inflammatory cytokine. Treg can be thymically derived or induced in peripheral tissues (Trams, L., et al., Curr. Top. Microbiol. Immunol. 293:115-131, 2005; Walker et al., J. Clin. Invest. 112:1437-1443, 2003; Bluestone, J., et al., Nat. Rev. Immunol. 3:253-257, 2003). The absence of Treg or their depletion leads to multisystemic autoimmunity in both mice and humans (Wildin, R., et al., Nat Genet. 27:18-20, 2001), while adoptive transfer of natural Treg prevents and cures autoimmune disease (Huter, E., et al., Eur. J. Immunol. 38:1814-1821, 2008; Tang, Q., et al., J. Exp. Med. 199:1455-1465, 2004).

CD4+CD25+ Treg cells are known to be present in both humans and mice and are characterized by expression of intracellular signaling molecule FoxP3 (for review, see Sakaguchi et al., Immunol. Rev. 182:18-32, 2001). Treg cells isolated from human peripheral blood are highly differentiated memory cells based on their FACS staining characteristics and short telomere length and historically are thought to be derived from the thymus (Taams et al., Eur. J. Immunol. 32:1621-1630, 2002; Jonuleit et al., J. Exp. Med. 193:1285-1294, 2001). In humans, Tregs are believed to represent <10% of all CD4+ T cells and require activation to induce suppressor function. The suppressive function of these Treg cells is mediated via cell-cell contact and is abrogated by the addition of IL-2 (Baecher-Allan et al., J. Immunol. 167:1245-1253, 2001). Tregs are known also to mediate suppression through production of IL-10 (Sakaguchi et al., Immunol. Rev. 182:18-32, 2001).

The Treg population is reduced in autoimmune-prone animals and humans (see Salomon et al., Immunity 12:431-440, 2000; Kukreja et al., J. Clin. Invest. 109:131-140, 2002). Mice carrying the X-linked scurfy mutation develop a multi-organ autoimmune disease and lack conventional CD4+CD25+ regulatory T cells (Fontenot et al., Nat. Immunol. 4:330-336, 2003; Khattri et al., Nat. Immunol. 4:337-342, 2003). It has been shown that the gene mutated in these mice is FoxP3, which encodes a member of the forkhead/winged helix family and acts as a transcriptional repressor (Schubert et al., J. Biol. Chem. 276:37672-37679, 2001). In mice, FoxP3 has been shown to be expressed exclusively in CD4+CD25+ Treg cells, and is not induced upon activation of CD25-cells. However, when FoxP3 is introduced via retrovirus or via transgene expression, naïve CD4+CD25− T cells are converted to Treg cells (Hori et al., Science 299:1057-1061, 2003). In humans, it has been noted that mutations in FoxP3 lead to a severe lymphoproliferative disorder known as IPEX (immunodysregulation, polyendocrinopathy, enteropathy, X-linked) syndrome, characterized by lymphoproliferative disease, insulin-dependent diabetes, thyroiditis, eczema, and death at an early age (see Wildin et al., J. Med. Genet. 39:537-545, 2002).

Due to their low frequency in peripheral blood, freshly isolated human CD4+CD25+ T cells with suppressive function are difficult to isolate and expand. In the autoimmune NOD mouse model, in which mice are transgenic for a single T cell receptor, investigators have isolated naturally occurring antigen-specific Treg cells from mouse spleen and lymph nodes, expanded the cells and demonstrated that transfer of these cells to the diabetic prone NOD mouse can suppress the development of diabetes (Tang et al., J. Exp. Med. 199:1455-1465, 2004; Masteller et al., J. Immunol. 175:3053-3059, 2005; Tarbell et al., J. Exp. Med. 199:1467-1477, 2004). This approach demonstrates the therapeutic benefit of Treg transfer to treat autoimmune disease. However, the approach used in the NOD mouse model is not therapeutically applicable to human subjects, due to the requirement that a large number of rare CD4+CD25+ T cells (approximately 4% of circulating T cells) be isolated from the peripheral blood. Further, this mouse model contains a single fixed T cell receptor (TCR) and does not address the problem of following TCR repertoire evolution or identifying antigen-specific T cells in complex systems where a polyclonal T cell response is present. Similar studies have not been possible in human subjects due to the low frequency of antigen-specific Treg cells circulating in the peripheral blood, especially with respect to autoreactive T cells.

Immunosuppression is desirable in many clinical settings, yet the ability to induce immune tolerance in an antigen-specific and/or site-specific manner is quite limited. In general, the immunosuppressant agents currently available require systemic administration and induce immunosuppression of a relatively non-specific nature. The drug toxicities and incidence of opportunistic infections resulting from the use of such non-specific immunosuppressant agents is unacceptably high.

Given the important role CD4+CD25+ regulatory T cells play in immune tolerance, there is a need to develop methods and compositions for generating regulatory CD4+CD25+ T cells for use in the treatment and/or prevention of autoimmune diseases, inflammatory conditions and for the prevention of graft rejection in a recipient following solid organ or stem cell transplantation. There are significant advantages to using the body's own mediators of immune tolerance, including CD4+CD25+FoxP3+ regulatory T cells (“Treg”), in order to supplant or supplement the use of pharmacologic immunosuppressants. However, thus far there are no workable methods known for inducing Treg for use in clinical applications. Although protocols exist for the in vitro induction of Treg from naïve T cell precursors, the toxic effects of systemic administration of such reagents limits their clinical utility.

There is also a great need for therapeutic agents to induce immune tolerance in airway inflammatory diseases. Approximately 32 million people suffer from airway inflammatory diseases including allergies and asthma in the U.S. (National Healthcare Disparities Report, 2009). Although desensitization protocols exist for specific allergens, these protocols are arduous and inefficient given that many individuals have multiple allergic triggers. Consequently, there is a need for therapeutic agents for use in treating airway inflammatory disease, such as therapeutic agents capable of inducing immune tolerance, such as by induction of TR1 cells.

### SUMMARY

In accordance with the foregoing, in one aspect, the present invention provides a composition for treating an airway inflammatory disease comprising (i) high molecular weight, crosslinked hyaluronan, and (ii) at least one aeroallergen. In some embodiments, the composition is formulated as an inhalable solution or an intranasal solution. In some embodiments, the inhalable solution comprises water or saline solution. In some embodiments, the inhalable solution or intranasal solution comprises hyaluronan at a concentration of from 0.001% to 10% (w/v). In some embodiments, the aeroallergen is selected from the group consisting of pollen, spores, mold, animal dander and dust mites. In some embodiments, the high molecular weight hyaluronan and at least one aeroallergen are subjected to a crosslinking agent after dilution in the saline solution.

In another aspect the invention provides a method of inducing immune tolerance to one or more aeroallergens in a mammalian subject suffering from or at risk for developing an airway inflammatory disease, the method comprising administering a composition comprising high molecular weight, crosslinked hyaluronan to a mammalian subject in an amount effective to induce immune tolerance to one or more aeroallergens in the mammalian subject. In some embodiments, the composition further comprises at least one aeroallergen. In some embodiments, the composition is formulated as an inhalable solution comprising water or saline solution which is administered to an airway of the subject. In some embodiments, the composition is formulated as an intranasal solution which is administered to a nasal passageway of the subject. In some embodiments, the inhalable solution is administered to the subject with a nebulizer. In some embodiments, the inhalable solution comprises hyaluronan at a concentration of from 0.001% to 10% (w/v). In some embodiments, the aeroallergen is selected from the group consisting of pollen, spores, mold, animal dander, and dust mites. In some embodiments, the mammalian subject is a human subject. In some embodiments, the human subject is suffering from or at risk for developing an airway inflammatory disease or condition of the lungs selected from the group consisting of: asthma, allergy, chronic obstructive pulmonary disease (COPD), emphysema, autoimmune lung disease, smoke inhalation, acute respiratory distress syndrome (ARDS), cystic fibrosis, laryngitis, bronchitis, pharyngitis, tracheitis, pneumonia, and sinusitis.

In another aspect the invention provides a method for treating a human subject suffering from an airway inflammatory disease comprising administering to the subject a composition comprising high molecular weight, crosslinked hyaluronan in an amount effective to induce immune tolerance to at least one aeroallergen in the human subject. In some embodiments, the composition further comprises at least one aeroallergen. In some embodiments, the composition is formulated as an inhalable solution comprising water or saline solution which is administered to an airway of the subject. In some embodiments, the composition is formulated as an intranasal solution which is administered to a nasal passageway of the subject. In some embodiments, the inhalable solution is administered to the subject with a nebulizer. In some embodiments, the inhalable solution comprises hyaluronan at a concentration of from 0.001% to 10% (w/v). In some embodiments, the human subject is suffering from or at risk for developing an airway inflammatory disease or condition of the lungs selected from the group consisting of: asthma, allergy, chronic obstructive pulmonary disease (COPD), emphysema, autoimmune lung disease, smoke inhalation, acute respiratory distress syndrome (ARDS), cystic fibrosis, laryngitis, bronchitis, pharyngitis, tracheitis, pneumonia, and sinusitis.

In another aspect, the invention provides an immunomodulatory hydrogel. The immunomodulatory hydrogel comprises hyaluronan and at least one T cell induction agent selected to induce a T cell response.

In another aspect, the invention provides a method of making an immunomodulatory hydrogel. The method comprises crosslinking a composition comprising hyaluronan, heparan sulfate and at least one T cell induction agent selected to induce a T cell response.

In another aspect, the invention provides a method for inducing a population of CD4+CD25+ regulatory T cells. The method comprises contacting a population of CD4+CD25− T cells with an immunomodulatory hydrogel comprising hyaluronan, heparan sulfate, and at least one T cell induction agent under conditions suitable to induce the population of CD4+CD25+ regulatory T cells.

In yet another aspect, the invention provides a method for inducing CD4+CD25+ regulatory T cells at or about a site of interest in a mammalian subject. The method comprises implanting an immunomodulatory hydrogel into a mammalian subject at a site of interest, wherein the immunomodulatory hydrogel comprises hyaluronan, heparan sulfate and at least one T cell induction agent selected to induce immunosuppressant regulatory CD4+CD25+ T cells that are FoxP3 positive.

In another aspect, the invention provides a composition comprising (i) high molecular weight, crosslinked, hyaluronan, (ii) heparin or heparan sulfate, and (iii) IL-10.

In another aspect, the invention provides a composition comprising (i) high molecular weight, crosslinked, hyaluronan, (ii) heparin or heparan sulfate, and (iii) at least one cytokine.

In another aspect, the invention provides a method of making a composition for treating an airway inflammatory disease, the method comprising crosslinking a solution comprising high molecular weight hyaluronan and heparan sulfate or heparin. In some embodiments, the solution further comprises IL-10.

In another aspect, the invention provides a method of inducing immunostimulatory T cells, such as TR1 cells, at or about a site of interest in a mammalian subject. The method according to this aspect of the invention comprises administering to the mammalian subject at a site of interest, a composition comprising high molecular weight, crosslinked hyaluronan and heparan sulfate or heparin in an amount effective to induce immunostimulatory T cells in the mammalian subject. In some embodiments, the composition further comprises IL-10.

In another aspect, the invention provides a method for treating an airway inflammatory disease or condition of the lungs in a mammalian subject in need thereof, the method comprising administering to the subject a composition comprising high molecular weight, crosslinked hyaluronan, heparan sulfate or heparin and IL-10 in an amount effective to induce immunostimulatory T cells, such as TR1, in the mammalian subject.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 illustrates a representative immunomodulatory hydrogel (10) comprising immunomodulatory agents in accordance with an embodiment of the invention, as described in Example 1;

FIG. 2 graphically illustrates the fold change in the percentage of CD4+ T cells that were GFP/FoxP3+ after incubation in the presence of soluble TGF-beta and IL-2 or in the presence of an HA/HS hydrogel comprising TGF-beta and IL-2, as described in Example 1;

FIG. 3A shows intracellular IL-10 staining following plate based activation with plate bound anti-CD3 antibody, soluble anti-CD28 antibody, IL-2 (20 IU/ml) and PBS (control), as described in Example 4;

FIG. 3B shows intracellular IL-10 staining following activation in the presence of High MW HA (1.5×10⁶ Da) (Genzyme), plus soluble anti-CD28 antibody and soluble IL-2 (20 IU/ml) and plate-bound anti-CD3 antibody, as described in Example 4;

FIG. 3C shows intracellular IL-10 staining following activation in the presence of HA/COL hydrogel (Extracel®) modified with the addition of streptavidin and biotinylated anti-CD3 antibody prior to polymerization, plus soluble anti-CD28 antibody and soluble IL-2 (20 IU/ml), as described in Example 4;

FIG. 3D shows intracellular IL-10 staining following activation in the presence of HA/HS/COL hydrogel (Extracel-HP™), modified with the addition of streptavidin and biotinylated anti-CD3 antibody prior to polymerization, plus soluble anti-CD28 antibody and soluble IL-2 (20 IU/ml), as described in Example 4;

FIG. 3E graphically illustrates the levels of TH1, TH2, and TH17 cytokines upon hydrogel based activation (n=3 experiments), as described in Example 4;

FIG. 4A shows the intracellular IL-10 staining of donor cells (CD45.2) and recipient cells (CD45.1) harvested four days after the CD45.2 donor cells had been previously embedded in an immunomodulatory hydrogel and injected into the recipient mouse, as described in Example 5;

FIG. 4B shows the intracellular IL-10 staining of donor cells (CD45.2) remaining embedded in the hydrogel four days after injection into the recipient mice in comparison to T cells harvested from the spleen of recipient mice (CD45.1), as described in Example 5;

FIG. 5A graphically illustrates IL-10 intracellular staining in CD45.2 cells harvested from the spleen 4 days after injection of the CD45.2 donor cells that had been previously embedded in an immunomodulatory hydrogel and injected into the recipient mouse, as described in Example 5;

FIG. 5B graphically illustrates IL-10 intracellular staining in CD45.1 cells harvested from the spleen 4 days after injection of the CD45.2 donor cells, as described in Example 5;

FIG. 5C graphically illustrates IL-10 intracellular staining in CD45.2 cells harvested from mesenteric lymph nodes 4 days after injection of the CD45.2 donor cells that had been previously embedded in an immunomodulatory hydrogel and injected into the recipient mouse, as described in Example 5;

FIG. 5D graphically illustrates IL-10 intracellular staining in CD45.1 cells harvested from the mesenteric lymph nodes 4 days after injection of the CD45.2 donor cells, as described in Example 5;

FIG. 5E graphically illustrates IL-10 intracellular staining in CD45.2 cells harvested from pancreatic lymph nodes 4 days after injection of the CD45.2 donor cells that had been previously embedded in an immunomodulatory hydrogel and injected into the recipient mouse, as described in Example 5;

FIG. 5F graphically illustrates IL-10 intracellular staining in CD45.1 cells harvested from pancreatic lymph nodes 4 days after injection of the CD45.2 donor cells, as described in Example 5;

FIG. 6A shows the results of FACS analysis of the population of cells harvested from the remaining hydrogel 4 days after implantation, gated for CD4 and CD45.2, showing the CD4+CD45.2+ (donor cells) and CD4+CD45.1 (recipient cells), as described in Example 6;

FIG. 6B shows the results of FACS analysis of the population of cells harvested from the remaining hydrogel 4 days after implantation, gated for CD4 and stained for CD4+CD45.2+ (donor cells) and GFP/FoxP3, as described in Example 6;

FIG. 6C shows the results of FACS analysis of the population of cells harvested from the spleen of the recipient animal 4 days after implantation of the hydrogel, gated for CD4 and stained for CD45.2, showing the CD4+CD45.2+ (donor cells) and CD4+CD45.1 (recipient cells), as described in Example 6;

FIG. 6D shows the results of FACS analysis of the population of cells harvested from the spleen of the recipient animal 4 days after implantation of the hydrogel, gated for CD4 and stained for CD4+CD45.2+ (donor cells) and GFP/FoxP3, as described in Example 6;

FIG. 7A shows the histological appearance of an islet within a pancreas, with the insulin-producing Beta cells stained darkly for the marker glucagon (see arrow pointing to Beta cells), as described in Example 7;

FIG. 7B is an image of a histological section stained for the marker glucagon (see arrow pointing to Beta cells) taken from a transplanted islet from an animal 10 days after receiving a FoxP3 inducing hydrogel together with an islet/bead construct, as described in Example 7;

FIG. 7C is an image of a histological section stained for the marker glucagon taken from a transplanted islet from an animal 10 days after receiving the islet/bead construct alone, as described in Example 7;

FIG. 8 graphically illustrates the slow release of IL-10 from HH-10 (crosslinked HA/HS and IL-10) over time in cell culture media, as described in Example 8;

FIG. 9 shows the results of FACS analysis after gating was performed on cells harvested from mice implanted with crosslinked HA/HS/COL or crosslinked COL control to sort the CD45.2+ (donor) and CD45.2-CD45.1+ (recipient) cells, as described in Example 9;

FIG. 10A shows the results of FACS analysis of IL-10 positive spleen donor cells (CD45.2+) obtained from mice that were implanted with (i) the crosslinked HA/HS/Col hydrogel, or (ii) the crosslinked COL control gel in comparison to spleen cells from the isotype control mice, as described in Example 10;

FIG. 10B shows the results of FACS analysis of IL-10 positive spleen recipient cells (CD45.1+) obtained from mice that were implanted with (i) the crosslinked HA/HS/Col hydrogel, or (ii) the crosslinked COL control gel in comparison to spleen cells from the isotype control mice, as described in Example 10;

FIG. 11A shows the results of FACS analysis of IL-10 positive mesenteric lymph node (LN) donor cells (CD45.2+) obtained from mice that were implanted with (i) the crosslinked HA/HS/Col hydrogel, or (ii) the crosslinked COL control gel in comparison to spleen cells from the isotype control mice, as described in Example 10;

FIG. 11B shows the results of FACS analysis of IL-10 positive mesenteric lymph node (LN) recipient cells (CD45.1+) obtained from mice that were implanted with (i) the crosslinked HA/HS/Col hydrogel, or (ii) the crosslinked COL control gel in comparison to spleen cells from the isotype control mice, as described in Example 10;

FIG. 12A graphically illustrates the results of FACS analysis analyzing intracellular IL-10 staining of CD4+CD62L-GFP/FoxP3− memory T cells that were activated with anti-CD3/CD28 antibodies and IL-2 for 96 hours in the absence (top panel, PBS control) or in the presence of 1% XHA (bottom panel), as described in Example 11;

FIG. 12B graphically illustrates the concentrations of cytokines IL-4, IL-6, IL-10, IL-17A, TNFα, and IFNγ in CD4+CD62L-GFP/FoxP3− memory T cells that were activated with anti-CD3/CD28 antibodies and IL-2 for 96 hours in the absence (PBS control) or presence of 1% XHA (N=4 experiments), as described in Example 11;

FIG. 13A illustrates the timeline of events in the mouse model of airway hypersensitivity wherein ovalbumin (OVA), a chick egg protein, serves as an antigenic trigger, used to demonstrate that XHA ameliorates airway hypersensitivity in a mouse model, as described in Example 11;

FIG. 13B graphically illustrates FACS analysis of lymphocytes, gated for CD4+ T-cells, that were isolated at Day 26 from DO.11 Rag−/− mice carrying a TCR specific for OVA that were treated with or without XHA according to the protocol shown in FIG. 13A, stained for intracellular IL-10 and KJI-26, an antibody clone that recognizes the DO.11 Ova-specific TCR (data is representative of two experiments), as described in Example 11;

FIG. 13C graphically illustrates the total cells in bronchoalveolar lavage (BAL) fluid obtained at Day 26 from conventional Balb/C mice that were treated with PBS control, OVA alone, or OVA+0.1% XHA according to the protocol shown in FIG. 13A, as described in Example 11; and

FIG. 13D graphically illustrates the number of eosinophils in bronchoalveolar lavage (BAL) fluid obtained at Day 26 from conventional Balb/C mice that were treated with PBS control, OVA alone, or OVA+0.1% XHA according to the protocol shown in FIG. 13A, as described in Example 11.

### DETAILED DESCRIPTION

Unless specifically defined herein, all terms used herein have the same meaning as they would to one skilled in the art of the present invention. The following definitions are provided in order to provide clarity with respect to the terms as they are used in the specification and claims to describe the present invention.

As used herein, the term “regulatory T cells” or “Treg” cells refers to T cells which express the cell surface markers CD4+ and CD25+, which express FoxP3 protein as measured by a Western blot and/or FoxP3 mRNA transcript.

As used herein, the term “Type 1 regulatory T cells” or “TR1” cells refers to T cells which express the cell surface marker CD4+, which are FoxP3−, and which mediate immune tolerance to self and foreign antigens via the production of prodigious amounts of IL-10.

As used herein, the term “hydrogel” refers to a water-insoluble polymeric material, which contains at least 10 percent water (by weight) when fully saturated.

As used herein, the term “heparan sulfate” (“HS”) refers to a class of glycosaminoglycans (GAG) characterized by a linear polymer of repeating dissacharide units comprising a glucuronic or iduronic acid residue and a glucosamine residue that are variously modified by O-sulfation, N-acetylation and/or N-sulfation. Included within the class of heparan sulfate is the mast cell product heparin (“HI”), which is more heavily sulfated. Heparan sulfate (HS) and heparin (HI) are closely related members of the glycosaminoglycan family of carbohydrates. Both HS and HI consist of a variably sulfated repeating disaccharide unit. The types of disaccharide units within HS and HI are shared but with some differences in the percent representation of particular disaccharides. For example, Glucuronic acid (GlcA) linked to N-acetylglucosamine (GlcNAc) typically makes up about 50% of the total disaccharide units in HS. In Heparin (HI), IdoA(2S)-GlcNS(6S) makes up about 75% of the disaccharide units from porcine intestinal mucosa. However, HI has GlcNAc content as well. In Gallagher, J., et al., Biochem. J. 230:665-674, 1985, it has been suggested that a HS-type glycosaminoglycan (GAG) should qualify as heparin only if its content of N-sulfate groups largely exceeds that of N-acetyl groups and the concentration of O-sulfate groups exceeds those of N-sulfate.

As used herein, the term “MHC Class II/peptide complex” refers to a complex comprising a peptide having an amino acid sequence that is cognate (e.g., identical or related to) at least one antigen in the induction culture. Any form of MHC Class II/peptide complex capable of binding T cells specific for the cognate antigen is intended to be within the scope of the present invention, including monomer, dimer, and multimer (e.g., tetramer) forms of MHC/peptide complexes.

As used herein, the term “antigen-specific regulatory T cells” or “antigen-specific Tregs” refers to Treg cells that were induced in the presence of an antigen and which express the cell surface markers CD4+ and CD25+, which express FoxP3 protein as measured by a Western blot and/or FoxP3 mRNA transcript. In an in vitro proliferation assay, after re-exposure to the cognate antigen used for induction, antigen-specific regulatory T cells are capable of actively suppressing the proliferation of freshly isolated CD4+CD25− T responder cells that have been stimulated in culture with an activating signal.

As used herein, the term “suppressor function” refers to the ability of a Treg cell to suppress the level of proliferation of a freshly isolated CD4+CD25− responder T cell population in a co-culture in response to an antigen as compared to the proliferation of CD4+CD25− in response to the antigen without the Treg cells, as measured in an in vitro assay.

As used herein, the term “responder T cell,” or “R,” refers to freshly isolated CD4+CD25− T cells that normally proliferate in response to an activating signal.

As used herein, the term “tolerance” includes refractivity to activating receptor-mediated stimulation. Such refractivity is generally antigen-specific and persists after exposure to the tolerizing antigen has ceased. For example, tolerance is characterized by lack of cytokine production, e.g., IL-2. Tolerance can occur to self antigens or to foreign antigens.

As used herein, the term “stimulatory molecules capable of binding to heparan sulfate or heparin,” refers to cytokines, chemokines, growth factors, and antibodies that possess a sequence of positively amino acids structurally capable of interacting with heparan sulfate or heparin. Exemplary stimulatory molecules capable of binding to heparan sulfate or heparin include, but are not limited to, Interleukins such as IL-2, IL-6, TGF-beta, IL-10, and antibodies such as anti-CD3 and anti-CD28.

As used herein, the term “self-antigen” refers to an immunogenic antigen or epitope which is native to a mammal and which may be involved in the pathogenesis of an autoimmune disease.

As used herein, the term “derived from” or “a derivative thereof,” in the context of peptide or polypeptide sequences, means that the peptide or polypeptide is not limited to the specific sequence described, but also includes variations in that sequence, which may include amino acid additions, deletions, substitutions, or modifications to the extent that the variations in the listed sequence retain the ability to modulate an immune response.

As used herein, the term “peptide” or “polypeptide” is a linked sequence of amino acids and may be natural, recombinant, synthetic, or a modification or combination of natural, synthetic, and recombinant.

As used herein, the expression “therapeutically effective amount” refers to an amount of the composition that is effective to achieve a desired therapeutic result, such as, for example, the prevention, amelioration, or prophylaxis of an autoimmune disease or inflammatory condition.

As used herein, an “autoimmune disease” is a disease or disorder arising from and directed against an individual's own tissues. Examples of autoimmune diseases or disorders include, but are not limited to, arthritis (rheumatoid arthritis, juvenile rheumatoid arthritis, psoriatic arthritis), conditions involving infiltration of T cells and chronic inflammatory responses, autoimmune myocarditis, multiple sclerosis, pemphigus, Type 1 diabetes (also referred to as insulin-dependent diabetes mellitus (IDDM)), and autoimmune lung disease.

As used herein, the term “organ or tissue transplant” refers to any solid organ such as kidneys, heart, lungs, liver, and pancreas, including tissue grafts, and whole or selected populations of blood or bone marrow transplants.

As used herein, the term “a mammalian subject suffering from clinical Type 1 diabetes (T1D)” (also referred to as insulin-dependent, juvenile diabetes, or childhood-onset diabetes), refers to a subject suffering from an autoimmune disease that results in destruction of insulin-producing beta cells of the pancreas, eventually resulting in a lack of insulin production. Symptoms of Type 1 diabetes include excessive excretion of urine (polyuria), thirst (polydipsia), constant hunger, weight loss, vision changes and fatigue. See World Health Organization (WHO) website. Conditions associated with T1D include hyperglycemia, hypoglycemia, ketoacidosis, and celiac disease. Complications associated with T1D include heart disease (cardiovascular disease), blindness (retinopathy), nerve damage (neuropathy), and kidney damage (nephropathy). See the American Diabetes Association website.

As used herein, the term “a mammalian subject suffering from or at risk for developing an airway inflammatory disease or condition of the lungs,” refers to a subject, such as a human subject, suffering from or at risk for developing asthma, allergy, chronic obstructive pulmonary disease (COPD), emphysema, autoimmune lung disease, smoke inhalation, acute respiratory distress syndrome (ARDS), cystic fibrosis, laryngitis, bronchitis, pharyngitis, tracheitis, phenumonia, or sinusitis.

As used herein the term “treating” or “treatment” refers to preventing, repressing, or eliminating at least one symptom associated with clinical Type 1 diabetes or an airway inflammatory disease or condition. Preventing at least one symptom associated with Type 1 diabetes or an airway inflammatory disease or condition involves administering a treatment to a subject prior to onset of the symptoms associated with clinical disease. Repressing at least one symptom associated with Type 1 diabetes or airway inflammatory disease or condition involves administering a treatment to a subject after clinical appearance of the disease or condition.

As used herein, the term “inhalable solution” refers to a solution that comprises at least 90% water or buffered saline (by weight) and is aerosolizable with standard equipment (e.g., a nebulizer).

In one aspect, the present invention provides an immunomodulatory hydrogel. The immunomodulatory hydrogel comprises hyaluronan and at least one T cell induction agent selected to induce a T cell response. In some embodiments, the immunomodulatory hydrogel further comprises heparan sulfate. In some embodiments, the immunomodulatory hydrogel further comprises collagen. The immunomodulatory hydrogel compositions of the present invention may be used to modulate (i.e., suppress or stimulate) an immune response in a mammalian subject.

In some embodiments, the immunomodulatory hydrogel compositions comprise T cell induction agents selected to generate CD4+CD25+FoxP3+ regulatory T cells in order to locally suppress an undesired immune response in a mammalian subject. The use of immunomodulatory hydrogels to provide a localized immunosuppressive effect is superior to generalized suppression in many disease settings in a mammalian subject (e.g., a human subject) in which localized (i.e., site-specific), and/or antigen-specific immune tolerance is preferable. The immunomodulatory hydrogels of the present invention may be used to treat tissue specific autoimmune and immune-mediated diseases, such as Type 1 diabetes, Rheumatoid arthritis, Celiac Sprue, Psoriasis, atopic diseases, allergy, Systemic Lupus Erythematosis (SLE), Multiple Sclerosis, Uveirtis, Scleroderma, Autoimmune Thyroiditis (Graves' disease), Sjogren's Syndrome, Herpes Keratosis, autoimmune hearing loss, autoimmune hepatitis, autoimmune myocarditis, Sialidenitis, Sarcoid, Graft versus Host Diseases (GVHD), Vitiligo, Crohn's disease, Ulcerative Colitis, and Ankylosing Spondylitis.

In some embodiments, the immunomodulatory hydrogel compositions comprise T cell induction agents selected to induce T cells to express IL-10, as described in Examples 4 and 5 herein.

The immunomodulatory hydrogels may also be used to facilitate cell-based transplant protocols (e.g., pancreatic islet cell transplantation, stem cell transplantation), tissue, and solid organ transplants.

### Immunomodulatory Compositions (Hydrogels or Solutions) Comprising Hyaluronan (HA) and Heparan Sulfate (HS)

In some embodiments, the immunomodulatory hydrogels of the present invention comprise hyaluronan, heparan sulfate, and at least one T cell induction agent, wherein the hydrogel is crosslinked with a crosslinking agent.

In some embodiments, the immunomodulatory compositions of the present invention comprising hyaluronan, heparan sulfate, or heparin and at least one cytokine, such as IL-10, are formulated into solutions, such as solutions suitable for delivery to an airway (e.g., inhalable solutions).

Hyaluronan (HA)

HA is an extracellular matrix (ECM) component comprised of repeating disaccharides, N-acetylglucosamine, and D-glucuronic acid. It is long, ranging in molecular weight from 10⁴ to 10⁷ Da, highly charged, and can bind large amounts of water (Laurent, T. C., et al., FASEB J. 7:2397-2404, 1992). Consequently, HA is of substantial structural importance in mammalian tissues where it serves as a space filler and a lubricant (Brown, T. J., et al., Exp. Physiol. 76:125-134, 1991). HA is highly biocompatible. Soluble HA is FDA approved for a wide variety of cosmetic and medical indications including filling out facial wrinkles, viscosupplementation of joint spaces, and a variety of ophthalmic indications (Kogan, G. L., et al., Biotechnol. Lett. 29:17-25, 2007). However soluble HA rapidly diffuses and degrades. To improve its stability and clinical efficacy, HA may be crosslinked into a hydrogel (Vercruysse, K. P., et al., Crit. Rev. Therapeut. Carrier Syst. 15:513-555, 1998). Alternatively, a solution containing HA may be crosslinked, such as an inhalable solution containing at least 90% water or saline (by weight).

In a preferred embodiment, the immunomodulatory hydrogels and solutions comprise high molecular weight HA. HA is known to have immunoregulatory properties. High molecular weight HA (HMW-HA) (>2,000 saccharides and >400 kDa) provides scaffolding for tissue repair in injury, is antiangiogenic and anti-inflammatory. Low molecular weight HA fragments (LMW-HA) (<16 saccharides and <3 kDa) are generated during infection and injury through the action of hyaluronidases, and can promote angiogenesis and proinflammatory responses. HA is the primary natural ligand for the extracellular matrix receptor CD44. It has been determined by the present inventors that CD44 crosslinking by high-molecular weight HA promotes expression of FoxP3, whereas LMW-HA does not (Bollyky, P. L., et al., J. Immunol. 183:2232-2241, 2009). As described by the present inventors, HMW-HA actively promotes immune tolerance by augmenting CD4+CD25+ Treg function, and LMW-HA does not (Bollyky, P. L., et al., J. Leukocyte Biology 86:1-6, 2009). In some embodiments, the hydrogel or solution comprises thiol-modified HA, such as found in the hydrogel commercially available as Extracel® (Glycosan Biosystems). The Extracel-HP™ Hydrogel kit contains Heprasil® (a combination of thiol-modified hyaluronan, HA, and thiol-modified heparin), Gelin-S® (thiol-modified gelatin), and Extralink® (a thiol-reactive crosslinker, polyethylene glycol diacrylate, PEGDA).

Heparan Sulfate and Heparin

Heparan sulfate (HS) comprises negatively-charged sulfo groups on the heparan chain Like HA, HS is also a linear polysaccharide that is ubiquitous in the human body and therefore non-antigenic. Unlike HA, HS is variably sulfated and consequently has the capacity to bind to variety of stimulatory molecules via non-covalent association with a sulfate group. Heparin (HI) is a structurally identical molecule to heparan sulfate except that it is hypersulfated. In some embodiments, Heparin is used in the immunomodulatory hydrogel formulations and solutions in order to permit maximal binding of stimulatory molecules. The incorporation of HS into HA hydrogels and solutions vastly expands the repertoire of molecules that can be delivered using a hydrogel or solution and allows for local, controlled-release delivery of a variety of growth factors and cytokines (Gallagher, J. T., et al., Proteoglycans: Structure, Biology and Molecular Interactions, Marcel Dekker Inc., New York, pp. 27-59, 2000). Positively-charged cytokines and growth factors, such as IL-2, IL-10 and/or TGF-beta, have clusters of positively-charged based amino acids that can form ion pairs with the negatively charged sulfo groups on the heparan chain.

Additional Conjugating Agents Included in the Hydrogels that are Capable of Binding to T Cell Induction Agents

In some embodiments, the immunomodulatory hydrogel further comprises one or more conjugating agent(s) capable of binding to at least one T cell induction agent, such as an antibody conjugating agent or an antigenic polypeptide conjugating agent. The various conjugating agents that may be used in accordance with this embodiment share the attribute of tethering the agent in question (via either a covalent or non-covalent attachment) to the hydrogel while leaving the agent functionally available to the appropriate T cell receptor.

Exemplary conjugating agents capable of binding T cell induction agents (e.g., cytokines, growth factors or antibodies) for use in the hydrogel include streptavidin in conjunction with biotinylated T cell induction agents. In another embodiment, agents are conjugated to beads made out of inert material such as polyethylene glycol (PEG) or to other polymers. The beads with conjugated agents are then suspended in the gels. In another embodiment, the agents are conjugated to an HS interacting protein (HIP) sequence, which allows the molecule to bind directly to the HS in the hydrogel (Liu et al., Journal of Biol. Chem. 273:9718-9726, 1998). In another embodiment, antibodies are chemically conjugated to HA, HS, or another polymer, which is then incorporated into the hydrogel. In another embodiment, in order to further retard the diffusion of the conjugated T cell induction agent(s), the biocompatible polylactic acid-60-glycolic acid (PGLA) could be added to hydrogel formulations (Pan, C. J., et al., J. Mater. Sci. Med. 18:2193-2198, 2007).

### Crosslinking Agents

The immunomodulatory hydrogels or solutions comprising HA and HS and at least one T cell induction agent are crosslinked using any suitable crosslinking agent. The crosslinking of the components that form the hydrogel (i.e., HA, HS and T cell induction agent(s)) may be carried out via non-chemical processes such as radiation treatment (i.e., electron beams, gamma rays, x-rays, ultraviolet light), or via chemical crosslinking processes such as cross-linking with a biscarbodiimide, protein cross-linking, and internal esterification (HAACP). For example, commercially available cross-linked HA preparations include Incert® (crosslinked with a biscarbodiimide) and Synvisc® or Restylane® (protein cross-linked).

Derivatives providing for covalently crosslinked networks are present in one embodiment of the present invention. An exemplary hydrogel matrix derived from a water-soluble, UV crosslinkable polymer comprises poly(ethylene glycol) dimethylacrylate (PEGDMA). This is used in conjunction with the conjugation of thiol groups to the glycosaminoglycans in question which are crosslinked by PEGDMA.

In some embodiments, the HA, HS and at least one cytokine, such as IL-10, are diluted into an inhalable solution prior to crosslinking, as described in more detail herein.

### Preferred Embodiments of the HA/HS Immunomodulatory Hydrogels

In some embodiments, the immunomodulatory hydrogels comprise from about 0.02% to about 20% w/v HA, (such as from 0.02% to about 10% HA, such as from about 0.02% to about 2% w/v HA) and from about 0.02% to about 2% w/v HS. In a preferred embodiment, the immunomodulatory hydrogels comprise from about 1% to about 0.1% w/v HA and from about 1% to about 0.1% w/v HS. In some embodiments, both HA and HS are present at a concentration of from about 1 μg/ml to about 10 μg/ml in the gel substrate prior to polymerization.

### T Cell Induction Agents

The immunomodulatory hydrogels of the present invention comprise at least one T cell induction agent selected to induce a T cell response, such as T cell polarization. In some embodiments, the hydrogel comprises a T cell induction agent selected to induce immunosuppressant regulatory CD4+CD25+ T cells that are FoxP3 positive. In some embodiments, the hydrogel comprises a T cell induction agent selected to induce polarization and/or differentiation of T cells into other T cell subsets.

Agents Selected to Generate Tregs:

Treg cells are a specialized subpopulation of T cells that have been shown to suppress CD4+ T cell, CD8+ T cell, NKT cell, and B-cell responses and thereby maintain immune homeostasis (Sakaguchi, S., Annu. Rev. Immunol. 22:531-562, 2004). Transfer of Treg cells is sufficient to protect from or reverse autoimmunity while an absence of Treg leads to severe, multisystemic autoimmune disease (Sakaguchi, S., 2004, supra). Treg cells are thought to mediate immune tolerance via a number of mechanisms, including immunosuppression via cytokines such as TGF-beta and IL-10 and contact-dependant granzyme or perforin-dependent killing of “suppressed” cells (von Boehmer, H., Nat. Immunol. 6:338-344, 2005).

The suppressive capacity of Treg has been demonstrated to correlate with the level of expression of the transcription factor FoxP3 (Sakaguchi, S., Annu. Rev. Immunol. 22:531-562, 2004). It is thought that Foxp3 may function as a transcriptional repressor, potentially through interactions with NF-AT and NF-κB (Schubert, L. A., et al., J. Biol. Chem. 276:37672-37679, 2001). Treg cells have a critical requirement for exogenous IL-2. While IL-2 promotes the proliferation and survival of all T cells, Treg cells are unique in that under most circumstances they are unable to produce this cytokine themselves (Sakaguchi, S., Annu. Rev. Immunol. 22:531-562, 2004). This requirement for IL-2 fits with the model of Treg cells as a regulatory cell type which exist within inflammatory contexts to dampen overaggressive immune responses.

Treg were originally thought to be exclusively derived from the thymus, but it has now been shown that they can be generated in the periphery and ex vivo (Taams, L. S., et al., Curr. Top. Microbiol. Immunol. 293:115-131, 2005; Walker, M. R., et al., J. Clin. Invest. 112:1437-1443, 2003; Bluestone, J. A., et al., Nat. Rev. Immunol. 3:253-257, 2003). The most consistent results in both mouse and human models have been achieved with a regimen consisting of TGF-beta, high-dose IL-2 and a potent TCR signal (Chen, W., et al., J. Exp. Med. 198:1875-1886, 2003; Zheng, S. G., et al., J. Immunol. 178:2018-2027, 2007). It is thought that TGF-beta and IL-2 promote FoxP3 induction through effects on SMAD3 and STATS signaling, respectively (Tone, Y., et al., Nat. Immunol. 9:194-202, 2008). Adoptive transfer of polyclonal Treg generated in this manner has been shown to treat or prevent development of autoimmunity in several animal models (Huter, E. N., et al., Eur. J. Immunol. 38:1814-1821, 2008; Selvraj, R. K., et al., J. Immunol. 180:2830-2838, 2008; Su, H., et al., Br. J. Dermatol. 158:1197-1209, 2008), including diabetes (Tang, Q., et al., J. Exp. Med. 199:1455-1465, 2004). Human but not mouse T cells can be induced to express FoxP3 upon activation in the setting of a potent TCR signal and ample IL-2 (Walker, M. R., et al., Proc. Natl. Acad. Sci. USA 102:4103-4108, 2005). However, such cells do not uniformly exhibit suppressive function (Gavin, M. A., et al., Proc. Natl. Acad. Sci. USA 103:6659-6664, 2006).

As demonstrated in Examples 1, 2, 6, and 7, the present inventors have discovered that the induction of FoxP3 positive CD4+CD25+ cells from CD4+CD25− cells occurred with enhanced efficiency in the presence of an immunomodulatory HA/HS hydrogel comprising anti-CD3 antibody, IL-2 and TGF-beta, as compared to a Matrigel® or fibrin gel control. Accordingly, in one embodiment, the HA/HS immunomodulatory hydrogel comprises at least one of an anti-CD3 antibody, and/or anti-CD28 antibody, and one or more cytokines, such as IL-2 and/or TGF-beta.

As demonstrated in Examples 4 and 5, the present inventors have discovered that the induction of IL-10 expression from CD4+CD25− cells occurred in the presence of an immunomodulatory HA/HS hydrogel comprising anti-CD3 antibody, anti-CD28 antibody and IL-2. Accordingly, in one embodiment, the HA/HS immunomodulatory hydrogel comprises at least one of an anti-CD3 antibody and/or anti-CD28 antibody, and one or more cytokines, such as IL-2 and/or IL-10. The HA/HS immunomodulatory hydrogel capable of inducing IL-10 according to this embodiment is useful to treat diseases or conditions in which IL-10 induction is beneficial, such as, for example, the treatment of subjects suffering from colitis. The co-infusion of regulatory T cells has been demonstrated to abrogate colitis in an IL-10 dependent manner (Asseman, C., et al., J. Exp. Med. 190:995-1004, 1999; Groux, H. A., et al., Nature 389:737-742, 1997). Other diseases which are known to improve upon treatment with IL-10 include animal models of diabetes (Slavin, A. J., Int. Immunol. 13:825-833, 2001), multiple sclerosis (Yang, J., et al., J. Clin. Invest. 119:3678-3691, 2005), and celiac disease (Salvati, V. M., et al., Gut 54:46-53, 2005).

In another embodiment, the HA/HS immunomodulatory hydrogel comprises at least one of an anti-CD3 antibody and/or anti-CD28 antibody and one or more cytokines, selected from the group consisting of IL-2, TGF-beta and IL-10.

In some embodiments, the T cell induction agents are selected to induce an antigen-specific T cell response, and further include at least one of antigenic proteins or peptides derived therefrom, or artificial MHC/peptide complexes. Whole antigenic proteins, portions thereof, or antigenic peptides may be added to the hydrogel components prior to polymerization. In some embodiments, the antigen is bound to a conjugating agent included in the hydrogel in order to avoid immediate diffusion of the antigen. The antigenic protein included in the hydrogel may be a self-antigen associated with an inflammatory or autoimmune pathology, or the antigenic protein may be chosen to control an undesirable immune response (e.g., to avoid transplant rejection).

In some embodiments, the choice of the antigenic peptide from among the amino acids comprising the antigenic protein depends in part on the binding properties of the MHC Class II type of a subject to be treated with implantation of the immunomodulatory hydrogel, the particular disease of interest, and the interactions of specific amino acids derived from an antigenic protein with a T cell receptor. In accordance with some embodiments of the present invention, the antigenic protein and peptide derived therefrom is chosen in reference to the MHC Class type of the subject. The MHC Class II type for the sample in question may be determined using standard techniques, such as, for example, an SSO based typing method (e.g., HLA-DRB and HLA-DQB SSO typing kits from Dynal Biotech LLC, Brown Deer, Wis.) or using sequence based HLA typing methods. Alternatively, the MHC Class II type of a particular subject may be obtained by referral to the subject's medical history.

In some embodiments, the chosen antigenic peptide is derived from a self-antigen. The self-antigen may be any tissue-specific antigen, including proteins known to be associated with, or found to be involved in, T cell-mediated disease, such as an autoimmune disease or an inflammatory condition. The self-antigen may be a protein or fragment, a variant, analog, homolog or derivative thereof. For example, an antigenic protein associated with the autoimmune disease Type 1 diabetes is glutamic acid decarboxylase (GAD), as further described in Example 3.

In other embodiments, the chosen antigenic peptide is derived from a foreign antigen. The foreign antigen may be any protein known to be associated with, or found to be involved in, T cell-mediated disease or inflammatory condition. For example, a foreign antigen may be expressed on allogeneic cells derived from a source other than the subject, such as, for example, in the context of transplantation (e.g., such as a solid organ transplant or bone marrow transplant). Alternatively, a foreign antigen may be added to the induction culture along with antigen presenting cells autologous to the source of T cells. The antigen-specific Treg cells generated using a hydrogel comprising a foreign antigen may be used to modulate an undesired T cell-mediated response against a foreign antigen.

The peptides derived from self-antigens or foreign antigens may be, for example, from about 9 to about 20 amino acids or more in length, more preferably about 9-10 amino acids in length. The peptides for use in the hydrogels of the invention may be prepared in a variety of ways. For example, peptides may be synthesized using an automated synthesizer (see, e.g., Hunkapiller et al., Nature 310:105-111, 1984; and Bodanszky, Principles of Peptide Synthesis, Springer Verlag, 1984). Alternatively, peptides may be synthesized by proteolytic cleavage (e.g., by trypsin, chymotrypsin, papain, V8 protease, and the like) or specific chemical cleavage (e.g., by cyanogen bromide). The peptides may also be synthesized by expression of nucleic acid sequences encoding a particular peptide.

In some embodiments, the T cell induction agent comprises one or more artificial MHC/peptide complexes. Any form of MHC Class II/peptide complex capable of binding T cells may be used in the methods of the present invention. For example, monomer, dimer, and multimer (e.g., such as tetramer) forms of MHC/peptide complexes may be used. MHC/peptide complex pools may also be used. In some embodiments, the MHC/peptide complexes are bound to a conjugating agent included in the hydrogel. The peptides present in the complex may be either covalently (e.g., by crosslinking or recombinant expression) or noncovalently attached to the MHC Class II molecules. Preferably, the peptide is non-covalently bound to the MHC molecules in the tetramer complex, thereby allowing more flexibility in the use of multiple types of peptides per construct, and also multiple types of peptide per tetramer. Therefore, a single MHC molecule can be loaded with a large number of distinct peptides. Methods of forming tetramers for use in the immunomodulatory hydrogels are described in U.S. Patent Application Publication No. 2003/0073102 A1, incorporated herein by reference, and can be adapted to form soluble tetramers for any desired HLA Class II molecule. The cDNA sequences of the various Class II HLA types are publicly available from Genbank. Further, the use of HLA Class II tetramers as a tool for binding antigen-specific T cells in other contexts is known in the art and various artificial HLA Class II type tetramers have been described. For example, the use of HLA-DQ tetramers is described in Kwok et al., J. Immunol. 164:4244-4249, 2000. The use of HLA DRA1 0101/DRB 0401 tetramers is described in Novak et al., J. Clin. Invest. 104:63-67, 1999.

Additional Components Added to the Hydrogel:

The immunomodulatory hydrogels may comprise additional components to enhance Treg induction, such as rapamycin, inhibitors of the signaling molecule ERK, and IL-2/II-2 antibody complexes, growth factors or immunosuppressants (Putnam, A. L., et al., Diabetes 58:652-662, 2009; Luo, X., et al., J. Immunol. 180:2757-2761, 2008). Such additional components may be bound to the hydrogel via a conjugating agent.

Agents Selected to Generate Other Immunomodulatory Subsets of T Cells

In other embodiments, the immunomodulatory hydrogel compositions comprise T cell induction agents selected to generate reactive T cells in order to enhance or skew an immune response. Depending on the particular cocktail of cytokines and other stimulatory factors inculcated into the hydrogel, several other subsets of T cells can be induced, as described below.

TH17 cells are a T cell subset thought to be important to immune responses to tumor cells and immune responses to fungal infections (Martin-Orozco et al., Immunity 31:787-798, 2009). Activation of T cells in the presence of TGF-beta and IL-6 is thought to drive differentiation of TH17 cells in the mouse (Weaver et al., Immunity 24:677-688, 2006), whereas in humans a combination of TGF-beta, IL-6, IL-1-beta and IL-23 is used (Manel, Nat. Immunol. 9:641-649, 2008). Accordingly, in one embodiment, the immunomodulatory hydrogel compositions comprise agents selected to induce TH17 cells comprising at least one of an anti-CD3 antibody, TGF-beta, IL-6, IL-1-beta, and IL-23, or a combination thereof. The immunomodulatory hydrogel compositions comprising agents selected to induce TH17 cells may be implanted at or near the site of the tumor for treatment of various cancers, such as, for example, breast cancer, prostate cancer, pancreatic cancer, lung cancer, ovarian cancer, colorectal cancer, stomach cancer, and melanoma.

TR1 cells are an immunoregulatory T cell subset thought to play an important role in immune regulation at sites of contact with microbes, such as the gut and lungs. Protocols for in vitro induction of TR1 cells call for IL-10 together with a TCR signal such as that delivered by anti-CD3 antibody (Roncarolo, Immunol. Rev. 212:28-50, 2006). Accordingly, in one embodiment, the immunomodulatory hydrogel compositions comprise agents selected to induce TR1 cells comprising at least one of an anti-CD3 antibody, anti-CD28 antibody, IL-2, IL-10, or a combination thereof. The immunomodulatory hydrogel compositions comprising agents selected to induce TR1 cells may be implanted at or near the site of the infection for treatment of various microbial infections.

As further described herein, IL-10 producing TR1 inhibit allergen-specific effector cells and promote immune tolerance in airway inflammation and colitis. Accordingly, in one embodiment, compositions capable of delivery to an airway (e.g., nasal passage and/or trachea), of a mammalian subject are provided comprising crosslinked HA, heparin, or heparan sulfate, and optionally IL-10. In some embodiments, the compositions are formulated in an inhalable solution for treatment of airway inflammation.

TH1 cells are a helper T cell subset thought to play a role in responses to viruses and intracellular bacteria. TH1 cells are also important in mitigating the effects of TH2 cells involved in most allergic and atopic processes. Protocols exist for the efficient in vitro induction of TH1 cells which call for the use of IL-12 and IL-2 in conjunction with a TCR signal such as that delivered by anti-CD3 antibody (Trincieri, Ann. Rev. Immunol. 13:251-276, 1995). Accordingly, in one embodiment, the immunomodulatory hydrogel compositions comprise agents selected to induce TH1 cells comprising at least one of an anti-CD3 antibody, IL-12, IL-2, or a combination thereof. The immunomodulatory hydrogel compositions comprising agents selected to induce TH1 cells may be implanted at or near the site of the infection or allergic or atopic condition for treatment of various viral or intracellular infections, or for the treatment of allergic or atopic conditions.

TH3 cells are a regulatory cell subset known to be important in oral tolerance. Protocols exist for the efficient in vitro induction of TH3 cells from T cell precursors using a combination of IL-10, IL-4, TGF-beta and anti-IL2 in conjunction with a TCR signal such as that delivered by anti-CD3 antibody (Faria and Weiner, Clin. Dev. Immunol. 13:143-157, 2006; Weiner, Immunologic Rev. 182:207-214, 2001).

In another aspect, a method is provided for inducing a population of CD4+CD25+ regulatory T cells. The method according to this aspect of the invention comprises contacting a population of CD4+CD25− T cells with an immunomodulatory hydrogel comprising hyaluronan, heparan sulfate, and at least one T cell induction agent.

In some embodiments, the method comprises contacting a population of CD4+CD25− T cells in a culture vessel in vitro with an immunomodulatory hydrogel in order to generate a population of CD4+CD25+ FoxP3 positive T cells. Mammalian T cells for use in this embodiment of the method of the invention may be isolated from a biological sample taken from a mammalian subject, such as a human subject, originating from a number of sources including, for example, peripheral blood mononuclear cells, bone marrow, thymus, tissue biopsy, tumor, lymph node tissue, gut associated lymphoid tissue, mucosa associated lymph node tissue, spleen tissue or any other lymphoid tissue and tumors. In a preferred embodiment, human T cells are isolated as peripheral blood mononuclear cells (PBMC) from a blood sample obtained from the peripheral blood of a subject. T cells may also be obtained from a unit of blood obtained from an apheresis or leukapheresis procedure.

In some embodiments, a population of CD4+CD25− T cells is included in the immunomodulatory hydrogel prior to polymerization, thereby creating an immunomodulatory hydrogel comprising embedded CD4+CD25− T cells, which may further comprise one or more T cell induction agents as described herein.

In some embodiments, a population of CD4+CD25+ T cells is included in the immunomodulatory hydrogel prior to polymerization, thereby creating an immunomodulatory hydrogel comprising embedded CD4+CD25+ T cells, which may further comprise one or more T cell induction agents as described herein.

A population of CD4+CD25− cells may be isolated from a sample comprising human T cells through the use of gradients and positive/negative selection techniques well known to those of skill in the art. For example, PBMC can be partially purified by density gradient centrifugation (e.g., through a Ficoll-Hypaque gradient), by panning, affinity separation, cell sorting (e.g., using antibodies specific for one or more cell surface markers, such as anti-CD4 and anti-CD25 antibodies), and other techniques that provide enrichment of CD4+CD25− cells. After selection, the enriched CD4+CD25− cell population is preferably at least 95% CD25−, more preferably at least 99% CD25−, more preferably at least 99.9% CD25−, up to 100% CD25−.

In some embodiments, the method further comprises contacting the cells in culture with a immunomodulatory hydrogel comprising at least one T cell induction agent selected to induce antigen-specific regulatory T cells. In such embodiments, antigen presenting cells autologous with the source of CD4+CD25− T cells may be added to the culture vessel to induce a population of antigen-specific CD4+CD25+ regulatory T cells. The antigen presenting cells or (“APCs”) may be any type of cell, such as, for example, dendritic cells or macrophages that are capable of taking up antigens, including antigenic peptides, processing them to small peptides and expressing them on their cell surface in the proper MHC Class II context for presentation to T cells. The antigen presenting cells may be autologous (e.g., derived from the subject), or the antigen presenting cells may be heterologous cells that are MHC matched to the source of CD4+ T cells. The methods according to this aspect of the invention may be used to generate Tregs for use as an immunotherapeutic agent to modulate an in vivo immune response to either a foreign or a self-antigen.

In another aspect, the invention provides a method for inducing CD4+CD25+ regulatory T cells at or about a site of interest in a mammalian subject. The method in accordance with this aspect of the invention comprises implanting an immunomodulatory hydrogel into a mammalian subject at a site of interest, wherein the immunomodulatory hydrogel comprises hyaluronan, heparan sulfate and a therapeutically effective amount of at least one T cell induction agent selected to induce immunosuppressant regulatory CD4+CD25+ T cells that are FoxP3 positive.

As used herein, the expression “therapeutically effective amount” refers to an amount of the hydrogel, and/or the amount of T cell induction agents included in the hydrogel, which is effective to achieve a desired therapeutic result, such as, for example, the prevention, amelioration or prophylaxis of Type 1 diabetes.

The immunomodulatory hydrogel comprising hyaluronan, heparan sulfate, and a therapeutically effective amount of at least one T cell induction agent is implanted in a mammalian subject in need thereof, such as a human, at a site appropriate to the disease to be treated and/or prevented. The implanted immunomodulatory hydrogels induce Treg cells locally in the environment surrounding the site of implantation in the human subject, which is useful in the context of a cellular therapy for regulating the immune response in the subject.

In some embodiments, the method comprises generating an immunomodulatory hydrogel comprising an embedded population of CD4+CD25− T cells obtained from the subject to be treated, wherein the hydrogel comprises one or more T cell induction agents as described herein.

In some embodiments, the method comprises generating an immunomodulatory hydrogel comprising an embedded population of CD4+CD25+ T cells obtained or derived from the subject to be treated, wherein the hydrogel comprises one or more T cell induction agents as described herein.

In some embodiments, the hydrogels may be used to induce Treg cells in order to prevent and/or treat a disease or condition such as an autoimmune disease. For many autoimmune diseases, the autoreactive antigens in question have been well characterized. The peptides or protein antigens in question may be conjugated to the hydrogels as described herein in order to induce antigen-specific Treg. This is also the case in transplant biology, where many histocompatibility antigens are well characterized and may be conjugated to immunomodulatory hydrogels as described herein in order to induce antigen-specific tolerance. An additional degree of specificity may be introduced by implanting the immunomodulatory hydrogels in proximity to the tissues in question (i.e., site-specific). For example, for autoimmune diseases such as Vitiligo, Type 1 diabetes, Autoimmune Thyroiditis, Ankylosing Spondylitis, Crohn's Disease, Psoriasis, and Rheumatoid Arthritis, the immunomodulatory hydrogels may be implanted in the skin, pancreas, thyroid, joints, peritoneum, skin, and joints, respectively. The immunomodulatory hydrogels may also be used in a site-specific manner to prevent rejection or inflammation directed at transplanted tissues.

In another embodiment, cells may be embedded into the immunomodulatory hydrogels. For example, tissues that do not form solid organs, but are nonetheless relevant to transplant biology, such as thyroid cells and insulin-producing pancreatic islets, may be embedded into the immunomodulatory hydrogels in order to foster localized Treg-induced immune tolerance to these tissues. In this context, the transplanted tissue (e.g., pancreatic islets) would serve as the source of antigen-specific stimulus.

Antigenic peptides useful for inclusion in the hydrogel used in the methods of the invention may be identified by eluting peptides from MHC molecules known to be associated with autoimmunity, for example, the HLA-DQ and DR molecules that confer susceptibility to several common autoimmune diseases such as Type 1 diabetes, rheumatoid arthritis and multiple sclerosis. Antigenic peptides useful in the present invention also include synthesized peptides predicted to bind to MHC molecules associated with autoimmune diseases.

In one embodiment, as described in Examples 3 and 7, the method of the invention may be used to generate antigen-specific Treg cells for treating and/or preventing Type 1 diabetes in those at risk for diabetes, by implanting an immunomodulatory hydrogel either at a site adjacent to the pancreas in the mammalian subject in need thereof, or at another location in the subject. As described in Examples 6 and 7, it has been determined that cells induced in the presence of the immunomodulatory hydrogel traffic to other parts of the body, such as the spleen. In the context of transplantation, the immunomodulatory hydrogel can be implanted into the subject prior to transplantation, at the time of transplantation, or after the transplantation.

In particular, those at risk for developing diabetes include first-degree relatives, and especially those individuals that have antibodies to islet-specific antigens. The methods of the invention can therefore be used to treat patients with active disease as well as prophylaxis for those identified (based on genetic or antibody screening) as being at risk for developing Type 1 diabetes.

Type 1 diabetes (T1DM) is an autoimmune disease mediated by the destruction of islet cells, the insulin-producing β-cells of the pancreas. This destruction represents a loss of immune tolerance and is due to pathogenic CD4+ and CD8+ T and B cell responses directed against proteins found in the islet. In the NOD mouse model, studies have demonstrated the ability to use islet specific Treg to protect and treat diabetes in several animal models (Tang et al., J. Exp. Med. 199:1455-1465, 2004; Tarbell et al., J. Exp. Med. 199:1467-1477, 2004).

In humans, several studies have identified abnormalities in the number or function of CD4+CD25+ Treg in patients with T1DM (Kukreja et al., J. Exp. Med. 199:1285-1291, 2004; Kriegel et al., J. Exp. Med. 199:1285-1291, 2004). A lack of Treg is also implicated in the pathogenesis of diabetes by the finding of diabetes in both animals depleted of Treg and in humans with IPEX (see Wildin et al., Nat. Genet. 27:18-20, 2001).

The MHC Class II molecules HLA-DQ8 and HLA-DQ-2, DRB1*0401, 0404, and DRB1*0301 confer the highest risk for individuals that have, or are at risk for Type 1 diabetes. Many islet-specific T cell auto-antigens have been identified that contribute to diabetes disease development (see, Masteller et al., J. Immunol. 171:5587-5595, 2003; Reijonen et al., Diabetes 51:1375-1382, 2002; Eisenbarth et al., Nat. Immunol. 3:344-345, 2002; and Maus et al., Clin. Immunol. 106:16-22, 2003), including glutamic acid decarboxylase (GAD), insulin, and IA2.

In another aspect, the present invention provides an immunomodulatory composition comprising (i) high molecular weight, crosslinked, hyaluronan, (ii) heparin or heparan sulfate and (iii) at least one or more stimulatory molecule(s) capable of binding to heparan sulfate or heparin comprising cytokines, chemokines, growth factors, and antibodies that possess a sequence of positively charged amino acids structurally capable of interacting with heparan sulfate or heparin. Exemplary stimulatory molecules capable of binding to heparan sulfate or heparin include, but are not limited to, Interleukins such as IL-2, IL-6, TGF-beta, IL-10, and antibodies such as anti-CD3 and anti-CD28. In some embodiments, the stimulatory molecule is a cytokine. In some embodiments, the composition comprises IL-10. In some embodiments of this aspect of the invention, the composition is suitable for delivery to the airway/respiratory tract of a mammalian subject for treatment of an airway inflammatory disease or disorder of the lungs. In some embodiments, the composition is formulated into a solution that is aerosolized using a nebulizer for delivery to an airway. In other embodiments, the composition is formulated into a solution that is delivered by lavage or injection into the sinuses or respiratory tract. In some embodiments, the composition is formulated into a preparation that is delivered to a subject by humidifier or vaporizer.

In some embodiments, the composition is formulated as an inhalable solution comprising water or phosphate buffered saline. In some embodiments, the inhalable solution comprises hyaluronan at a concentration of from 0.001% to 10% (w/v). In some embodiments, the inhalable solution comprises heparan sulfate at a concentration of from 0.001% to 10% (w/v). In some embodiments, the composition comprises IL-10 at a concentration of from 1 pg/ml to 1 mg/ml. In some embodiments, the inhalable solution comprises hyaluronan at a concentration of from 0.02% to 2% (w/v), heparan sulfate at a concentration of from 0.02 to 2% (w/v) and IL-10 at a concentration of from 1 pg/ml to 1 mg/ml.

In another aspect, the invention provides a method of making a composition for treating an airway inflammatory disease or condition, the method comprising crosslinking an inhalable solution comprising high molecular weight hyaluronan and heparan sulfate or heparin. In some embodiments, the inhalable solution comprises water or a saline solution. In some embodiments, the composition further comprises IL-10.

In some embodiments, the method comprises combining high molecular weight hyaluronan and heparan sulfate or heparin in a solution, followed by crosslinking the solution. For example, combining hyaluronan at a concentration of from 0.001% to 10% (w/v), and heparan sulfate or heparin at a concentration of from 0.001% to 10% (w/v) in a solution, followed by crosslinking the solution with a crosslinking agent. In some embodiments, the solution further comprises IL-10 at a concentration of from 1 pg/ml to 1 mg/ml.

The compositions and inhalable solutions comprising high molecular weight HA; HA/HS (or HA/HI); or HA/HS (or HA/HI) and at least one cytokine, such as IL-10; may be crosslinked using chemical or non-chemical crosslinking agents. For example, the crosslinking of hyaluronan and heparin or heparan sulfate can be carried out via a non-chemical process, such as radiation treatment (i.e., electron beams, gamma rays, x-rays, ultraviolet light); or via a chemical crosslinking process such as crosslinking with biscarbodiimide, protein crosslinking, or internal esterification (HAACP). For example, commercially available cross-linked hyaluronan preparations include Incert (crosslinked with a biscarbodiimide) and Synvisc® or Restylane® (protein cross-linked).

In one embodiment, derivatives providing for covalently crosslinked networks are present. An exemplary crosslinked matrix derived from a water-soluble, UV crosslinkable polymer comprises poly(ethylene glycol) dimethylacrylate (PEGDMA), which is used in conjunction with the conjugation of thiol groups to the glycosaminoglycans in question which are crosslinked by PEGDMA.

In some embodiments, high MW HA is solubilized in water or phosphate buffered saline (PBS) prior to crosslinking at a concentration of from 0.001% and 10% (w/v). The HA may be crosslinked prior to dilution in water or PBS, or the solution containing solubilized HA may be crosslinked.

In some embodiments, the high MW HA and HS (or HI) are crosslinked, either separately prior to combining, or in a solution comprising both HA and HS suitable for inhalation.

In one embodiment, high MW HA, HS (or HI) and IL-10 are combined in a solution suitable for inhalation at concentrations effective to treat airway inflammation, and the solution is then treated with a crosslinking agent. After crosslinking, the solution is administered to a subject for treatment.

The solution containing high MW crosslinked HA; HA/HS (or HA/HI); or HA/HS (or HA/HI) and IL-10; may be aerosolized with a nebulizer and delivered to the airway of a mammalian subject for use as a valuable, inexpensive and biocompatible therapy for treatment of allergies and asthma in accordance with the methods of the invention.

In another aspect, the invention provides a method of inducing immunostimulatory T cells, such as TR1, at or about a site of interest in a mammalian subject. The method according to this aspect of the invention comprises administering to the mammalian subject at a site of interest, a composition comprising high molecular weight, crosslinked hyaluronan and heparan sulfate (or heparin) in an amount effective to induce immunostimulatory T cells (e.g., TR1) in the mammalian subject. In some embodiments, the composition further comprises IL-10.

In some embodiments, the invention provides a method for treating an airway inflammatory disease or condition of the lungs in a mammalian subject in need thereof, the method comprising administering to the subject a composition comprising high molecular weight, crosslinked hyaluronan, heparan sulfate (or heparin) and IL-10 in an amount effective to induce immunostimulatory T cells in the mammalian subject.

In some embodiments, the method comprises administering to the subject a composition, such as an inhalable solution comprising hyaluronan at a concentration of from 0.001% to 10% (w/v). In some embodiments, the inhalable solution comprises heparan sulfate (or heparin) at a concentration of from 0.001% to 10% (w/v). In some embodiments, the composition comprises IL-10 at a concentration of from 1 pg/ml to 1 mg/ml. In some embodiments, the inhalable solution comprises hyaluronan at a concentration of from 0.02% to 2% (w/v), heparan sulfate (or heparin) at a concentration of from 0.02 to 2% (w/v) and IL-10 at a concentration of from 1 pg/ml to 1 mg/ml.

In some embodiments, the composition is formulated as an inhalable solution comprising water or saline solution which is administered to an airway of the subject. In some embodiments, the inhalable solution is aerosolized using a nebulizer and administered to the subject with an inhaler. In other embodiments, the composition is formulated into a solution that is delivered to the subject by lavage or injection into the sinuses or respiratory tract. In some embodiments, the composition is formulated into a preparation that is delivered to the subject by humidifier or vaporizer.

Formulations ready for use in the methods of the invention may be produced from concentrates, for example by the addition of isotonic saline solutions. Sterile formulations ready for use may be administered using nebulizers which produce inhalable aerosols by means of ultrasound, compressed air, compressed gases, or propellants.

In some embodiments, the methods are used to treat a human subject, suffering from or at risk for developing an airway inflammatory disease or condition of the lungs selected from the group consisting of: asthma, allergy, chronic obstructive pulmonary disease (COPD), emphysema, autoimmune lung disease, smoke inhalation, acute respiratory distress syndrome (ARDS), and cystic fibrosis.

As described in Example 11, the present inventors have further demonstrated that airway treatment with a composition comprising high molecular weight, cross-linked hyaluronan (referred to as “XHA”) in the presence of at least one ambient allergen (either present in the environment, or present in the composition, or both), such as an aeroallergen, promotes antigen-specific TR1 induction in vivo and ameliorates disease in a mouse asthma model. As demonstrated in Example 10, inhaled XHA creates a pro-tolerogenic environment that builds immune tolerance to allergens encountered or delivered to a mammalian subject in the context of XHA. This unique approach to inducing TR1 in a polyclonal manner has significant potential to augment the underlying mechanisms of asthma and other airway inflammatory diseases.

In accordance with the foregoing, in another aspect, the invention provides a composition for treating an airway inflammatory disease, the composition comprising (i) high molecular weight, crosslinked, hyaluronan, and (ii) at least one aeroallergen capable of triggering an allergic reaction in a mammalian subject.

As used herein, the term “aeroallergen” refers to an airborne substance which triggers an allergic reaction in a mammalian subject. In some embodiments, the aeroallergen is an ambient allergen present in the environment at sufficient concentrations to induce an airway inflammatory disease. Non-limiting examples of aeroallergens include pollen, spores, mold, animal dander and dust mites. In some embodiments, the composition comprises an aeroallergen selected from the group consisting of pollen, spores, mold, animal dander and dust mites.

In some embodiments, the composition is formulated as an inhalable solution. In some embodiments, the inhalable solution comprises water or phosphate buffered saline. In some embodiments the inhalable solution comprises hyaluronan at a concentration of from 0.001% to 10% (w/v).

In some embodiments, the composition is formulated as an intranasal solution which is administered to a nasal passageway of the subject.

The compositions, inhalable solutions and intranasal solutions comprising high molecular weight HA may be crosslinked using chemical or non-chemical crosslinking agents. In some embodiments, the solutions further comprise at least one aeroallergen and are crosslinked prior to, or after, addition of the at least one aeroallergen.

For example, the crosslinking of hyaluronan may be carried out via a non-chemical process, such as radiation treatment (i.e., electron beams, gamma rays, x-rays, ultraviolet light); or via a chemical crosslinking process such as crosslinking with biscarbodiimide, protein crosslinking or internal esterification (HAACP). For example, commercially available cross-linked hyaluronan preparations include Incert (crosslinked with a biscarbodiimide) and Synvisc® or Restylane® (protein cross-linked).

In one embodiment, derivatives providing for covalently crosslinked networks are present. An exemplary crosslinked matrix derived from a water-soluble, UV crosslinkable polymer comprises poly(ethylene glycol) dimethylacrylate (PEGDMA), which is used in conjunction with the conjugation of thiol groups to the glycosaminoglycans in question which are crosslinked by PEGDMA.

In some embodiments, the high molecular weight hyaluronan is subjected to a crosslinking agent prior to dilution in a saline solution, which optionally includes at least one aeroallergen.

In some embodiments, the high molecular weight hyaluronan and at least one aeroallergen are subjected to a crosslinking agent after dilution in the saline solution.

In another aspect, the invention provides a method of inducing immune tolerance to one or more aeroallergens in a mammalian subject suffering from or at risk for developing an airway inflammatory disease, the method comprising: administering a composition comprising high molecular weight, crosslinked hyaluronan to a mammalian subject in an amount effective to induce immune tolerance to one or more aeroallergens in the mammalian subject.

In some embodiments, the composition further comprises at least one aeroallergen. In some embodiments, the aeroallergen is an ambient allergen present in the environment of the mammalian subject. In some embodiments, the at least one aeroallergen is selected from the group consisting of pollen, spores, mold, animal dander and dust mites.

In some embodiments, the composition is formulated as an inhalable solution comprising water or saline solution which is administered to an airway of the subject. In some embodiments, the inhalable solution is administered to the subject with a nebulizer. In some embodiments, the inhalable solution comprises hyaluronan at a concentration of from 0.001% to 10% (w/v).

In some embodiments, the mammalian subject is a human subject suffering from or at risk for developing an airway inflammatory disease or condition of the lungs selected from the group consisting of: asthma, allergy, chronic obstructive pulmonary disease (COPD), emphysema, autoimmune lung disease, smoke inhalation, acute respiratory distress syndrome (ARDS), cystic fibrosis, laryngitis, bronchitis, pharyngitis, tracheitis, pneumonia and sinusitis.

In another aspect, the invention provides a method for treating a human subject suffering from an airway inflammatory disease comprising administering to the subject a composition comprising high molecular weight, crosslinked hyaluronan in an amount effective to induce immune tolerance to at least one aeroallergen in the human subject.

In some embodiments, the composition further comprises at least one aeroallergen. In some embodiments, the aeroallergen is an ambient allergen present in the environment of the mammalian subject. In some embodiments, the at least one aeroallergen is selected from the group consisting of pollen, spores, mold, animal dander, and dust mites.

In some embodiments, the composition is formulated as an inhalable solution comprising water or saline solution which is administered to an airway of the subject. In some embodiments, the inhalable solution is administered to the subject with a nebulizer. In some embodiments, the inhalable solution comprises hyaluronan at a concentration of from 0.001% to 10% (w/v).

In some embodiments, the composition is formulated as an intranasal solution and is administered to a nasal passageway of the subject.

In some embodiments, the mammalian subject is a human subject suffering from or at risk for developing an airway inflammatory disease or condition of the lungs selected from the group consisting of: asthma, allergy, chronic obstructive pulmonary disease (COPD), emphysema, autoimmune lung disease, smoke inhalation, acute respiratory distress syndrome (ARDS), cystic fibrosis, laryngitis, bronchitis, pharyngitis, tracheitis, pneumonia, and sinusitis.

### EXAMPLES

The following examples merely illustrate the best mode now contemplated for practicing the invention, but should not be construed to limit the invention.

### Example 1

This example describes the generation of a hydrogel capable of inducing FoxP3+ Regulatory T cells and demonstrates that the immunomodulatory hydrogels promote the induction of functional FoxP3+Regulatory T cells from naïve T cell precursors.

### Rationale

An immunomodulatory hydrogel was designed and constructed that was capable of delivering a set of costimulatory signals for inducing functional FoxP3+ regulatory T cells (Tregs) from naïve T cell precursors. The immunomodulatory hydrogel included crosslinked hyaluronan (HA) to provide structure and fluid retention to the hydrogel. HA provides an important and unexpected costimulatory signal to T cells which promotes regulatory function, as described in Bollyky, P. L., et al., J. Immunol. 179:744-747, 2007; Bollyky, P. L., et al., J. Immunol. 183:2232-2241, 2009; and Bollyky, P. L., et al., J. Leukocyte Biol. 86:567-572, 2009. HS was also included in the immunomodulatory hydrogel to bind cytokines and growth factors (e.g., IL-2) in a charge-dependent manner, thereby providing sequestration and presentation of these mediators to the infiltrating cells. IL-2 and TGF-beta were included in the hydrogel to provide costimulatory signals for induction of functional FoxP3+ regulatory T cells (Tregs) from naïve T cell precursors. In the embodiment of the immunomodulatory hydrogel described in this Example, streptavidin was added to the gel substrate prior to crosslinking as an antibody tethering agent to allow for incorporation of biotinylated anti-CD3 antibody into the hydrogel.

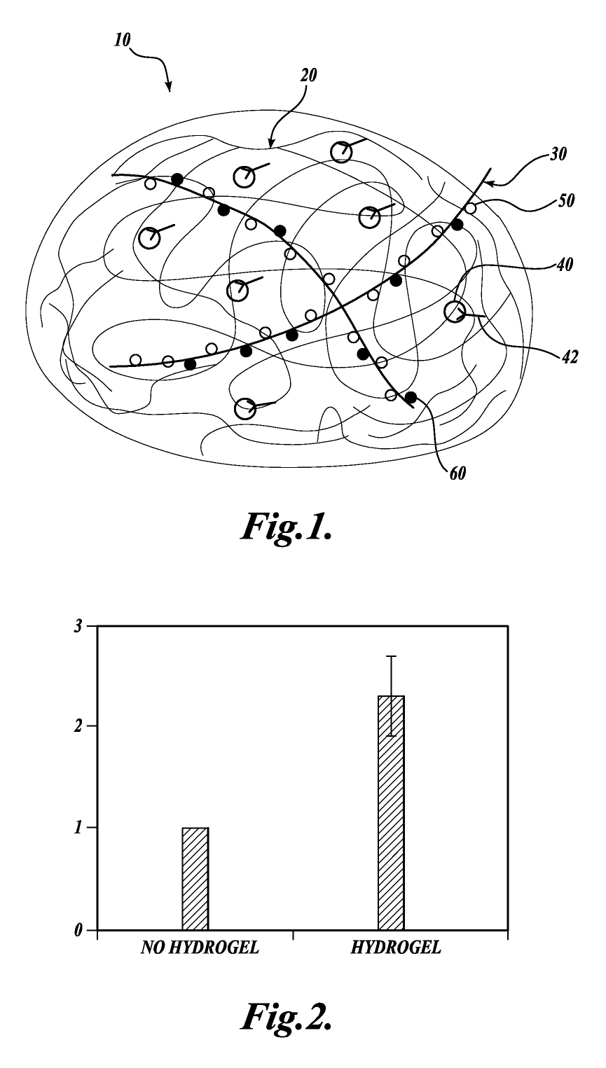

FIG. 1 illustrates a representative immunomodulatory hydrogel (10) comprising immunomodulatory agents in accordance with an embodiment of the invention. In the embodiment of the immunomodulatory hydrogel (10) shown in FIG. 1, the hydrogel (10) comprises HA (20), HS (30), and further comprises streptavidin (40) as an antibody conjugating agent, at least a portion of which is bound to biotinylated anti-CD3 antibodies (42). As further shown in FIG. 1, in some embodiments, the immunomodulatory hydrogel (10) may further include cytokines and growth factors such as TGF-beta (50) and IL-2 (60), which are reversibly (i.e., non-covalently) bound to the HS (30) in a charge-dependant manner.

### Methods

Generation of Immunomodulatory Hydrogels:

The commercially available hydrogel substrate Extracel-HP™ (Glycosan Biosystems) was used as a starting material. Extracel-HP™ is composed of Heprasil® (thiol-modified sodium hyaluronate (HA) with thiol-modified heparin (HS)), Gelin-S® (thiol-modified gelatin), Extralink® (PEGDA, polyethylene glycol diacrylate), and degassed, deionized water. Solutions of Heprasil® and Gelin-S® form a transparent hydrogel when mixed with the crosslinking agent Extralink®, a thiol-reactive crosslinker, polyethylene glycol diacrylate (PEGDA).

The Extracel® used in this experiment was prepared in accordance with the manufacturer's instructions in a 96 round bottom well plate, with a diameter of 6 mm. Briefly described, the Heprasil®, Gelin-S®, and Extralink® solutions were prepared by dissolving the lyophilized solids in deionized water. When reconstituted, the three materials were in 1× phosphate buffered saline (PBS), pH˜7.4. Within 2 hours of making the reconstituted solutions, equal volumes of Heprasil® and Gelin-S® were mixed. To form the hydrogel, Extralink® was added to the Heprasil® and Gelin-S® mix in a 1:4 volume ration (0.5 mL Extralink® to 2.0 ml Heprasil®+Gelin-S®) and mixed by pipette. Gelation occurred within about 20 minutes. Once hardened, the hydrogel was easily transferred or stored.

Immunomodulatory Hydrogels Comprising Immunomodulatory Agents:

For hydrogels comprising additional agents, the additional agents were added to the mixture of Heprasil® and Gelin-S® prior to adding the crosslinking agent. The crosslinking agent was then added and the gels were allowed to polymerize for 1 hour in a 96 well prior to use. Streptavidin was added at 10 μg/ml. Biotinylated anti-CD3 antibody was added at 10 μg/ml. TGF-beta was added at 10 ng/ml. Anti-CD28 antibody was added at 0.5 μg/ml.

Matrigel® (Control):

Matrigel® is a gel-like matrix produced by tumor cells. The bulk of Matrigel® is composed of laminin, which is notable for being an extracellular matrix molecule which does not bind to CD44. Matrigel® also contains smaller amounts of other molecules including collagen type IV, heparan sulfate proteoglycans, and entactin. While it is possible that Matrigel® may bind cytokines due to the presence of heparan sulfate, it is likely that the heparan sulfate is complexed with various proteoglycans, and therefore would not be expected to bind cytokines at a level that is biologically equivalent to the purified heparan sulfate present in the HA/HS hydrogel Extracel®.

Matrigel® was obtained from BD Pharminigen. Matrigel® was reconstituted and autopolymerized per the manufacturer's instructions. For Matrigel® comprising additional agents, the additional agents were added to the Matrigel® prior to autopolymerization. Streptavidin was added at 10 μg/ml. Biotinylated anti-CD3 antibody was added at 10 μg/ml.

Fibrinogen Gel (Control):

Fibrinogen scaffolds are commonly used in bioengineering. Fibrinogen is known to be a CD44 ligand, but fibrin does not bind cytokines. Fibrinogen (Invitrogen) was reconstituted at a concentration of 3 mg/ml and cross-linked with thrombin (Sigma) added at 1 U/ml. For fibrinogen comprising additional agents, the additional agents were added to the fibrinogen prior to polymerization. Streptavidin was added at 10 μg/ml. Biotinylated anti-CD3 antibody was added at 10 μg/ml.

The following types of hydrogels were prepared:
1. Hydrogel (HA/HS) with no additional agents (control).
2. Hydrogel (HA/HS) plus streptavidin (10 μg/ml) (Pierce Biotechnology) and biotinylated anti-CD3 antibody (10 μg/ml).
3. Hydrogel (HA/HS) plus streptavidin (10 μg/ml) (Pierce Biotechnology) and biotinylated anti-CD3 antibody (10 μg/ml), plus TGF-beta (10 ng/ml) plus anti-CD28 antibody (0.5 μg/ml).

Isolation of Naïve T Cells:

CD4+ cells were isolated from FoxP3-GFP C57BL/6 mice. In these animals the fluorescent marker GFP is transcribed in conjunction with FoxP3 and in fixed proportion to FoxP3 (Hori et al., Science 299:1057-1061, 2003; Fontenot et al., Immunity 22:329-341, 2005). There is generally an excellent agreement between GFP expression in these animals and FoxP3 levels, as demonstrated in Bollyky et al., J. Immunol. 183:2232-2241, 2009, incorporated herein by reference. The use of GFP/FoxP3 positive cells from these animals allows for accurate tracking of FoxP3 induction by virtue of their fluorescent tag without compromising the viability of the cells.

Mouse leukocyte populations were isolated from inguinal, axial, and brachial lymph nodes and spleen cells from 6 to 8 week old mice. CD4+ T cell populations were isolated using a CD4+ T cell isolation kit (Miltenyi Biotec), according to the manufacturer's instructions. T cells were then sorted into both FoxP3/GFP+ and FoxP3/GFP− fractions using a FACS-Vantage Flow Cytometer Cell Sorter. The isolated CD4+ cells were sorted using flow cytometry to deplete them of all GFP/FoxP3+ Treg cells. Thus, any FoxP3 expression observed was due to induction, and not the result of the proliferation of extant, naturally occurring Treg cell populations. Purity of the resulting cell fractions was reliably >99.9% FoxP3/GFP−.

Induction of Regulatory T Cells (Treg):

Cells were cultured in DMEM-10 (Invitrogen) supplemented with 10% FBS (Hyclone), 100 μg/ml penicillin, 100 U/ml streptomycin, 50 μM Beta-mercaptaethanol, 2 mM glutamine, and 1 mM Na Pyruvate (Invitrogen). 2×10⁵ naïve CD4+GFP/FoxP3− T cells were cultured in 200 μl media for 72 hours together with the following reagents: Note: for the hydrogel containing conditions, the hydrogels were added as a crosslinked hydrogel disc formed in a well of a 96 well plate, the hydrogel disc having a diameter of about 6 mm.
1. No hydrogel: Media: plate bound anti-CD3 antibody and soluble anti-CD28 antibody (1 μg/ml) added to the culture.
2. No hydrogel: plate bound anti-CD3 antibody and soluble anti-CD28 antibody (1 μg/ml) plus soluble TGF-beta, and soluble IL-2 added to the culture.
3. HA-HS hydrogel: crosslinked in presence of streptavidin-biotinylated anti-CD3 antibody; soluble anti-CD28 antibody (1 μg/ml) added to the culture.
4. HA-HS hydrogel: crosslinked in presence of streptavidin-biotinylated anti-CD3 antibody; recombinant TGF-beta, and recombinant IL-2; soluble anti-CD28 antibody (1 μg/ml) was also added to the culture.
5. Matrigel®: crosslinked in the presence of streptavidin-biotinylated anti-CD3 antibody; soluble anti-CD28 antibody (1 μg/ml) were then added to the hydrogels in solution and were allowed to bind to the hydrogels.
6. Matrigel®: crosslinked in the presence of streptavidin-biotinylated anti-CD3 antibody; recombinant TGF-beta, and recombinant IL-2; soluble anti-CD28 antibody (1 μg/ml) was also added to the culture.

Flow Cytometry Analysis:

After 72 hours in culture, flow cytometry analysis was carried out on the cells in culture using a fluorochrome-labeled antibody against CD25 (clone PC61.5, BD Biosciences). The following T cell activation antibodies were utilized: anti-CD3e (145-2C11, eBioscience) and anti-CD28 (37.51, eBioscience), according to the manufacturer's recommended protocols.

### Results

The results of one representative experiment carried out as described above are shown below in TABLE 1. The pooled data from four experiments is shown in FIG. 2.

TABLE 1
Effect of the Immunomodulatory Hydrogels on FoxP3 Induction

| | | CD25−/ | CD25+ | CD25− | CD25+ |
|---|---|---|---|---|---|
| | | FoxP3− | FoxP3− | FoxP3+ | FoxP3+ |
| No hydrogel | Media only | 9.9% | 90% | 0.1% | 0.4% |
| (plate-bound | control | | | | |
| anti-CD3 | Media plus | 4.0% | 83% | 0.1% | 13% |
| antibody) | TGF-beta and | | | | |
| | IL-2 | | | | |
| HA/HS | hydrogel only | 78% | 19% | 1.0% | 2.4% |
| hydrogel | hydrogel plus | 5.6% | 45% | 0.1% | 50% |
| (Extracel ®) | TGF-beta and | | | | |
| with | IL-2 | | | | |
| gel-bound | | | | | |
| anti-CD3 | | | | | |
| antibody | | | | | |
| Matrigel ® | Matrigel ® | 87% | 7.6% | 0.8% | 4.5% |
| (control) | only | | | | |
| mixed with | Matrigel ® | 55% | 35% | 0.4% | 9.6% |
| anti-CD3 | plus TGF-beta | | | | |
| antibody | and IL-2 | | | | |

As shown above in TABLE 1, FoxP3 induction was not observed under any conditions without the addition of IL-2 and TGF-beta. However, upon the addition of these cytokines, FoxP3 induction was observed to occur with enhanced efficiency in the presence of HA/HS hydrogel (Extracel) comprising gel-bound streptavidin-biotinylated anti-CD3 antibody, but not in the presence of Matrigel® polymerized in the presence of streptavidin-biotinylated anti-CD3 antibody, as shown in TABLE 1 and FIG. 2. FIG. 2 graphically illustrates the fold change in the percentage of CD4+ T cells that were GFP/FoxP3+ after incubation in the presence of soluble TGF-beta and IL-2 or in the presence of an HA/HS hydrogel comprising TGF-beta and IL-2. Results similar to the Matrigel®, (i.e., no FoxP3 induction) were also observed for fibrin gel (data not shown).

The enhanced efficiency of FoxP3 induction in the presence of HS/HS hydrogel comprising gel-bound streptavidin-biotinylated anti-CD3 antibody was not due to any obvious difference in the intensity of T cell receptor activation, and the increase in FoxP3 expression was present irrespective of the concentration of plate-bound CD3 used in the control sample (data not shown). It was also determined that there was no significant alteration in lymphocyte viability in the presence of the hydrogels over the period of the assay (data not shown).

The polyclonal Treg induction using the immunomodulatory hydrogels described in this example may be used for clinical applications in which antigen non-specific Treg are desired, such as, for example, solid organ transplantation and graft versus host disease (GVHD). Hydrogels capable of inducing polyclonal (non-antigen specific) Treg, while not antigen-specific, would nonetheless be useful for inducing tolerance in a local (i.e., site-specific) manner. This is because of the propensity of Treg cells activated in peripheral tissues to remain within the particular tissue distribution of their origin (Wheeler et al., J. Immunol. 183(12):7635-7638, 2009). For example, immunomodulatory hydrogels implanted in the peritoneum or skin are expected to be useful in the treatment of GVHD where the immune response is thought to be directed against a myriad of host and microbial agents and is typically site-specific to the skin and/or gut. Polyclonal Treg induction may also be used in autoimmune disorders characterized by multi-systemic autoimmunity, such as scleroderma, and generalized failures of immune tolerance, such as SLE.

### Example 2

This example demonstrates that treatment with heparanase and the exclusion of heparan sulfate from the hydrogel substrate diminishes the extent of FoxP3 induction observed in the presence of immunomodulatory hydrogels.

### Methods

Generation of Immunomodulatory Hydrogels:

HA/HS hydrogels were generated as described in Example 1, with the difference that prior to the polymerization step, Streptavidin (Pierce Biotechnology) and biotinylated anti-CD3 antibody were both added at 5 μg/ml. The hydrogels were allowed to polymerize in 96 well plates for 1 hour prior to use.

For the HA/HS hydrogel (Extracel-HP™) with heparanase condition, the gels were generated as described above, then were pretreated with heparanase (1 μg/ml) for 1 hour prior to use in cell culture.

For the HA hydrogel without Heparan Sulfate, the hydrogel was prepared as described above in Example 1, with the exclusion of Heparan Sulfate.

Isolation of Naïve T Cells:

Naïve CD4+GFP/FoxP3− T cells were obtained from FoxP3-GFP C57BL/6 mice as described above in Example 1.

Induction of Regulatory T Cells (Treg):

Naïve CD4+GFP/FoxP3− T cells were cultured in DMEM-10 (Invitrogen) supplemented with 10% FBS (Hyclone), 100 μg/ml penicillin, 100 U/ml streptomycin, 50 μM Beta-mercaptaethanol, 2 mM glutamine and 1 mM Na Pyruvate (Invitrogen). 2×10⁵ naïve CD4+GFP/FoxP3− T cells were cultured in 200 μl media for 72 hours together with the following reagents:
1. No Gel: plate bound anti-CD3 antibody; soluble recombinant IL-2 (Chiron) at 100 IU/ml, soluble recombinant TGF-beta (R&D Systems) at 10 ng/ml, and soluble anti-CD28 antibody (1 μg/ml) added to the culture.
2. HA/HS hydrogel (Extracel®): crosslinked in presence of streptavidin-biotinylated anti-CD3 antibody; soluble recombinant IL-2 (Chiron) at 100 IU/ml, soluble recombinant TGF-beta (R&D Systems) at 10 ng/ml, and soluble anti-CD28 antibody (1 μg/ml) added to the culture.
3. HA/HS hydrogel (Extracel®): crosslinked in presence of streptavidin-biotinylated anti-CD3 antibody (treated with heparanase); soluble recombinant IL-2 (Chiron) at 100 IU/ml, soluble recombinant TGF-beta (R&D Systems) at 10 ng/ml, and soluble anti-CD28 antibody (1 μg/ml) added to the culture.
4. HA (no HS) hydrogel: crosslinked in presence of streptavidin-biotinylated anti-CD3 antibody; soluble recombinant IL-2 (Chiron) at 100 IU/ml, soluble recombinant TGF-beta (R&D Systems) at 10 ng/ml, and soluble anti-CD28 antibody (1 μg/ml) added to the culture.

Flow Cytometry Analysis:

Cells were induced for 72 hours in the above culture conditions and analyzed by flow cytometry as described in Example 1.

### Results

The results of one representative experiment carried out as described above are shown below in TABLE 2.

TABLE 2
Effect of Immunomodulatory Hydrogels Pretreated With Heparanase,
or Hydrogels Excluding Heparan Sulfate, on FoxP3 Induction

| | CD25−/ | CD25+ | CD25− | CD25+ |
|---|---|---|---|---|
| | FoxP3− | FoxP3− | FoxP3+ | FoxP3+ |
| #1: No Gel | 3.4% | 69% | 0.04% | 28% |
| #2: HA/HS hydrogel | 4.7% | 48% | 0.04% | 48% |
| (Extracel ®): | | | | |
| #3: HA/HS hydrogel | 6.1% | 60% | 0.04% | 33% |
| (Extracel ®) treated with | | | | |
| heparanase | | | | |
| #4: HA hydrogel (no HS) | 58% | 27% | 0.40% | 15% |

As shown above in TABLE 2, cells activated with plate-bound anti-CD3 antibody together with soluble anti-CD28 antibody (Condition #1: No Gel), demonstrated de novo induction of FoxP3. However, superior FoxP3 induction was achieved using the HA/HS hydrogel with gel-bound anti-CD3 antibody (Condition #2). This enhanced FoxP3 induction was highly dependent upon the inclusion of Heparan Sulfate in the hydrogel, as both the heparanase treated HA/HS hydrogel (Condition #3) and hydrogel lacking Heparan Sulfate (Condition #4) resulted in a substantially diminished FoxP3 induction. These data indicate that the presence of HS enhances the capacity of IL-2 and TGF-beta to induce Treg.

### Example 3

This example describes the generation of immunomodulatory hydrogels capable of inducing antigen-specific regulatory T cells (antigen specific Tregs).

### Rationale

The delivery of FoxP3 induction cues locally and via slow diffusion for site-specific immunosuppression is desirable. Immunosuppression is desirable in many clinical settings, yet the ability to induce immune tolerance in an antigen-specific and/or site-specific manner is quite limited. In general, the immunosuppressant agents currently available require systemic administration and induce immunosuppression of a relatively non-specific nature. The drug toxicities and incidence of opportunistic infections resulting from the use of such non-specific immunosuppressant agents is unacceptably high. Diffusion kinetics have been described for a variety of growth factors, including TGF-beta (Cai, S., et al., Biomaterials 26:6054-6067, 2005; Pike, D. B., et al., Biomaterials 27:5242-5251, 2006).

This example describes exemplary methods for using an immunomodulatory hydrogel, generated as described in Examples 1 and 2, in a clinical setting, such as treatment of Type 1 diabetes. In one embodiment, an immunomodulatory hydrogel is implanted at a site in the subject's body (e.g., adjacent to pancreatic islet cells) such that it is capable of locally inducing antigen-specific regulatory T cells at the site of interest to provide site-specific immunosuppression. The site-specific immunosuppression can be either antigen-specific, or non-specific, depending on the immunomodulatory agents present in the hydrogel.

### Methods

Mouse Models:

As described in Examples 1 and 2, induction of CD4+CD25+ Treg that express FoxP3 (non-antigen specific) can be studied using CD4+ T cells isolated from GFP/FoxP3 knock-in mice that are subsequently depleted of GFP/FoxP3+ cells.

For antigen-specific FoxP3 induction, mouse models relevant to autoimmune disease can be used. For example, a first mouse model to study antigen-specific FoxP3 induction is the DR0401-GAD transgenic mouse. These are RAG−/− mice carrying a transgenic T cell receptor specific for a defined epitope of glutamate decarboxylase (GAD), an important target of autoantibodies in people who later develop Type 1 diabetes.

A second mouse model useful for studying antigen-specific FoxP3 induction comprises a pair of complementary mouse strains: RAG−/− mice carrying a DO11.10 T cell receptor transgene and RIP-OVA transgenic mice. The former possesses an OVA-specific T cell receptor, while the later expresses membrane bound ovalbumin under the control of the rat insulin promoter. As shown previously, transfer of activated DO11.10 T cells into RIP-OVA mice instigates development of autoimmune diabetes (J. Exp. Med. 199:1725-1730, 2004). Both DO11.10 and DR0401-GAD mice have been crossed against GFP/FoxP3 knock-in mice and offspring will be screened for use in the following experiments.

In conjunction with the above animal models, the present inventors possess peptides and tetramers specific to both ovalbumin as well as the relevant portion of GAD. These tools will allow the generation and tracking of antigen-specific responses together with FoxP3 expression in these animal models.

Immunomodulatory Hydrogels for FoxP3 Induction:

Immunomodulatory hydrogels for antigen-specific FoxP3 Treg induction comprise the same elements as shown in the hydrogel (10) illustrated in FIG. 1, including HA (20), HS (30) an antibody conjugating agent (40), at least a portion of which is bound to anti-CD3 antibodies (42). As further shown in FIG. 1, in some embodiments, the immunomodulatory hydrogel (10) may further include cytokines and growth factors such as TGF-beta (50) and IL-2 (60), which are reversibly bound to the HS (30) in a charge-dependant manner.

The hydrogels for antigen-specific FoxP3 induction further comprise at least one T cell induction agent that induces antigen-specific regulatory T cells, such as an antigenic protein or peptides derived therefrom. In one embodiment, a whole protein, such as ovalbumin or other whole protein, is incorporated into the hydrogel substrate prior to polymerization, similar to the method used to incorporate streptavidin described in Examples 1 and 2. Antigen presenting cells, such as dendritic cells, are provided naturally in vivo in the host. In embodiments in which the hydrogel is to be utilized to generate antigen-specific Treg cells in vitro, antigen presenting cells can be added to the culture system.

In another embodiment, for smaller proteins and peptides, the antigen is bound to a conjugating agent included in the hydrogel in order to avoid immediate diffusion of antigen. The antigen can be bound to the conjugating agent in the hydrogel using any suitable method of attachment. For example, a conjugating agent such as streptavidin, and antigen such as biotinylated MHC-peptide monomers or tetramers can be added to the hydrogel prior to polymerization. MHC-peptide complexes have been used to induce functional human Treg (Long et al., Eur. J. Immunol. 39:612-620, 2009). MHC-peptide complexes can be generated in biotinylated form using several methodologies, for example, as described in Yang et al., Hum. Immunol. 65:692-699, 2004.

In another example, the antigen is provided as a bifunctional peptide which contains the antigenic peptide in tandem with an HS interacting protein (HIP) sequence (Liu, S., et al., J. Biol. Chem. 273:9718-9726, 1998). This HIP sequence would allow the bifunctional peptide to bind directly to the HS in the hydrogel.

In another example, the MHC-peptide monomers are chemically conjugated to HA, HS, or another polymer which is then incorporated into the hydrogel. In another example, the MHC-peptide monomers are conjugated onto beads made out of an inert material such as polyethylene glycol (PEG). The PEG beads are then suspended in the hydrogels.

Assay for Regulatory Function of Tregs Induced with Immunomodulatory Hydrogels:

FoxP3 positive cells induced using the immunomodulatory hydrogel system described herein, from the GFP/FoxP3 knock-in mice, and the antigen-specific mouse models, are assayed for in vivo function by infusing the putative Treg into Scurfy mice that lack FoxP3, and the infused mice are evaluated for protection from lethal autoimmune disease. Given that IL-2 and TGF-beta in conjunction with a T cell receptor signal have reliably induced functional Treg in other induction protocols (Tone, Y., et al., Nat. Immunol. 9:194-202, 2008; Huter, E. N., et al., Eur. J. Immunol. 38:1814-1821, 2008); it is expected that the Treg cells induced using the immunomodulatory hydrogels will likewise be functional.

Assay for Generation of Immunomodulatory Hydrogels with Improved Function:

In order to study the impact of hydrogel immunomodulatory agents and concentrations on the IL-2 and TGF-beta signaling, the levels of pSTAT5 and pSMAD-3 levels are determined in cells after contacting the cells with the immunomodulatory hydrogels. It is expected that the levels of pSTAT5 and pSMAD-3 will be increased for hydrogel-bound IL-2 and hydrogel-bound TGF-beta as compared to soluble IL-2 and soluble TGF-beta. It is further expected that an increase in the level of sulfation of HS will correlate to an increase in induction efficiency. The amounts of hydrogel-bound cytokine versus cell surface bound radiolabeled cytokine will be determined.

Evaluation of Antigen-Specific Immunomodulatory Hydrogels Using In Vivo Models:

The RIP-OVA mouse model of autoimmune diabetes can be used to assess the ability of Treg cells to function in an antigen-specific manner in vivo that were induced using the immunomodulatory hydrogels.

### Methods

Treg are induced which specifically recognize ovalbumin starting with CD4+GFP/FoxP3− precursors taken from DO11.10 mice using the immunomodulatory hydrogel as described above. The activated DO11.10 T-effector cells (T cells depleted of FoxP3+ cells) are then adoptively transferred with or without OVA-specific DO11.10/FoxP3+induced Treg into RIP-OVA host mice. These host animals are then monitored for development of hyperglycemia. Periodically, the host animals will be sacrificed and evaluated histologically for OVA-targeted inflammation in the pancreas. Protection from diabetes in animals which receive coadministration of DO11.10/FoxP3+ induced Treg into RIP-OVA and diabetes inducing activated DO11.10 T-effector cells is indicative of successful antigen-specific Treg induction. In addition to monitoring disease progression, the migration and viability of the induced Treg in vivo will be made possible by virtue of their expression of GFP/FoxP3. This same marker will also allow the introduced, induced Treg cells to be distinguished from endogenous regulatory T cells in histologic sections.

In Vivo Implantation of Immunomodulatory Hydrogels:

Immunomodulatory hydrogels capable of delivering antigen-specific stimulus (e.g., GAD-specific, made as described herein), are implanted into the omentum of recipient mammalian subjects prior to the infusion of activated DO11.10 T-effector cells. The omentum is chosen for two reasons. First, the omentum is a well-vascularized space with lymphatics which drain to the same lymph nodes that serve the pancreas. Second, protocols for implantation of hydrogels into the omentum are well developed in the context of islet transplantation protocols (Kobayashi, T., et al., Cell Transplant. 15:359-365, 2006).

In another animal model, hydrogels capable of stimulating GAD-specific responses are implanted into the omentum of DR0401-GAD mice. While these animals do not develop diabetes, their use will allow for the evaluation of Treg induction using peptide antigens in another system with relevance to human diabetes.

### Example 4

This example demonstrates that incubation of T cell precursors in the presence of an immunomodulatory hydrogel stimulates IL-10 production in vitro.

### Rationale

Regulatory T cells promote immune suppression through the production of the immunosuppressive cytokine IL-10. IL-10 plays crucial roles in the induction of peripheral tolerance to self and foreign antigens by inhibiting antigen presentation and regulation of immune responses (Roncarolo, M. G., et al., Immunol. Rev. 212:28-50, 2006). Disorders in IL-10 production or signaling result in autoimmune disease (Asseman, C. S., et al. J. Exp. Med. 190:995-1004, 1999; Martinez-Forero, I. R., et al., Eur. J. Immunol. 38:576-586, 2008) and allergy (Wu, K., et al., Cell Mol. Immunol. 4:269-275, 2007). Conversely, adoptive transfer of IL-10 producing regulatory T cells has been shown to ameliorate autoimmunity and allergy in several animal models (Groux, H. A., et al., Nature 389:737-742, 1997; Slavin, A. J., Int. Immunol. 13:825-833, 2001; Salvati, V. M., et al., Gut 54:46-53, 2005).

Soluble HA is not suitable for clinical applications because it rapidly diffuses and degrades. To improve its stability and clinical efficacy HA is often crosslinked into a hydrogel. Extracel® is an HA and collagen (COL) based hydrogel preparation marketed for cell culture applications (Prestwich, G. D., et al., Adv. Exp. Med. Biol. 585:125-133, 2006; Zheng, S., et al., Biomaterials 25:1339-1348, 2004, both references hereby incorporated herein by reference). As described herein, in various embodiments, HA/COL hydrogel has been modified to generate an immunomodulatory hydrogel.

As described in Example 2, the presence of heparan sulfate (HS) in a HA containing hydrogel (HA/HS/COL), such as Extracel-HP™ enhances the capacity of IL-2 to induce Treg.

The following experiment was carried out to determine whether the incubation of T cell precursors in the presence of the immunomodulatory hydrogels stimulate IL-10 production.

### Methods

Conventional T cell precursors were incubated in cell culture under the following conditions:
1. Plate-bound anti-CD3 antibody, soluble anti-CD28 antibody, soluble IL-2 plus PBS (control);
2. High MW HA (1.5×10⁶ Da (Genzyme), plus soluble anti-CD28 antibody and soluble IL-2 (20 IU/ml) and plate-bound anti-CD3 antibody;
3. HA/COL hydrogel (Extracel) modified with the addition of streptavidin and biotinylated anti-CD3 antibody prior to polymerization, plus soluble anti-CD28 antibody and soluble IL-2 (20 IU/ml); and
4. HA/HS/COL hydrogel (Extracel-HP™) modified with the addition of streptavidin and biotinylated anti-CD3 antibody prior to polymerization, plus soluble anti-CD28 antibody and soluble IL-2 (20 IU/ml).

T cell precursors (2×10⁵ cells) were incubated in culture with 25 μl of the HA containing substrate. For the HA-based hydrogels, cells were layered on top of 25 μl volume of hydrogels following polymerization for this experiment. Where indicated, biotinylated anti-CD3 antibody (145-2C11, BD Biosciences) and streptavidin (Sigma Aldrich) were each added at 10 μg/ml prior to polymerization. Soluble CD28 antibodies were added at 1.0 μg/ml. IL-2 was added at 20 IU/ml.

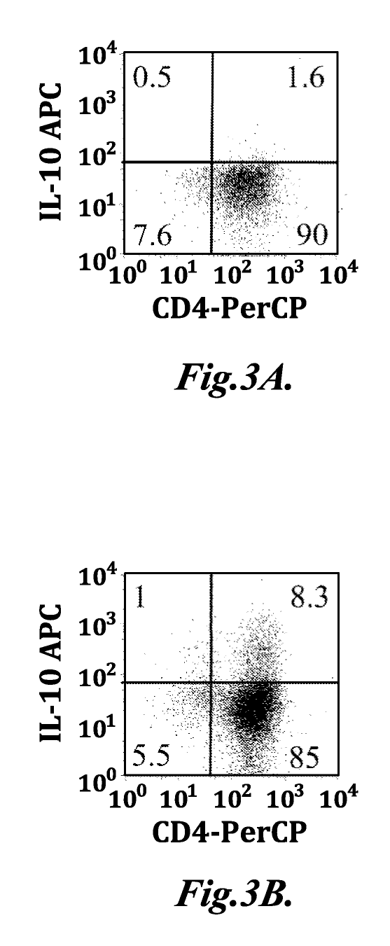

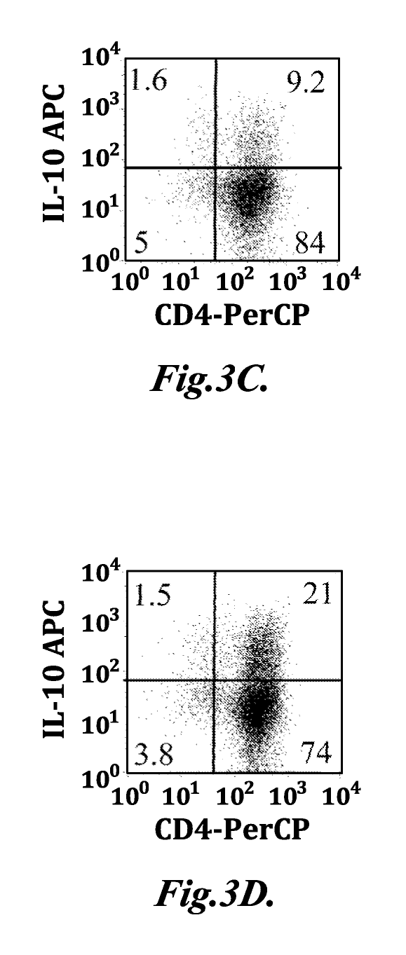

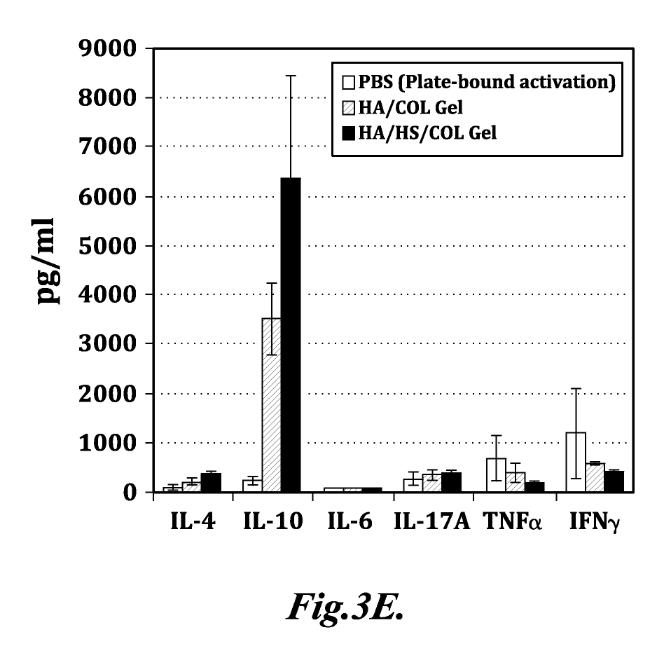

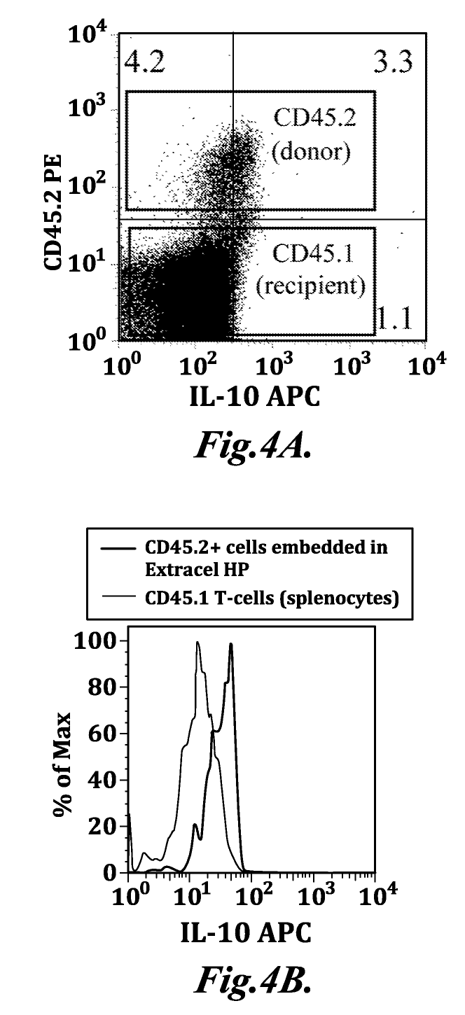

After 96 hours of incubation, the cell cultures were stained for intracellular IL-10 (FIG. 3). Concentrations of selected cytokines in the cell culture supernatants were also determined from cells incubated under the same conditions (n=4 independent experiments) (FIG. 4A). Analysis of cell culture supernatants for cytokines was performed via ELISA (BD Biosciences). Cytokine data was normalized to proliferation data by setting up parallel wells, which received (³H) thymidine and were analyzed as described in Bollyky, P. L, et al., J. Immunol. 183:2232-2241, 2009. The accompanying cytokine production values were then divided by the counts per minute (CPM) for the condition in question.

### Results

FIGS. 3A-D show intracellular IL-10 staining following plate based or hydrogel based activation (n=5 experiments). FIG. 3A shows intracellular IL-10 staining following plate based activation with plate bound anti-CD3 antibody, soluble anti-CD28 antibody, IL-2 (20 IU/ml) and PBS (control). FIG. 3B shows intracellular IL-10 staining following activation in the presence of High MW HA (1.5×10⁶ Da (Genzyme), plus soluble anti-CD28 antibody and soluble IL-2 (20 IU/ml) and plate-bound anti-CD3 antibody. FIG. 3C shows intracellular IL-10 staining following activation in the presence of HA/COL hydrogel (Extracel®) modified with the addition of streptavidin and biotinylated anti-CD3 antibody prior to polymerization, plus soluble anti-CD28 antibody and soluble IL-2 (20 IU/ml). FIG. 3D shows intracellular IL-10 staining following activation in the presence of HA/HS/COL hydrogel (Extracel-HP™) modified with the addition of streptavidin and biotinylated anti-CD3 antibody prior to polymerization, plus soluble anti-CD28 antibody and soluble IL-2 (20 IU/ml).

As shown in FIG. 3D, substantial IL-10 production was observed using the HA/HS/COL gel as a platform for cell culture in vitro. In contrast, as shown in FIG. 3A, IL-10 production was not observed using plate-bound anti-CD3 antibody and soluble anti-CD28 antibody and IL-2. It was determined that IL-10 production was not observed using the same streptavidin/antibody complex incorporated into either Matrigel® or a fibrin hydrogel (data not shown). As shown in FIG. 3, omission of the HA component of the hydrogel, but not the HS or collagen components, diminished IL-10 production.

FIG. 3E graphically illustrates the levels of TH1, TH2, and TH17 cytokines upon hydrogel based activation (n=3 experiments) in vitro.

These results demonstrate that incubation of T cell precursors in the presence of an immunomodulatory hydrogel stimulates IL-10 production in vitro.

### Example 5

This example demonstrates that an HA-based immunomodulatory hydrogel containing embedded T cells promotes IL-10 production in vivo.

### Rationale

In this example, the ability of an HA-based immunomodulatory hydrogel to induce IL-10 production by T cells was examined. A GFP/FoxP3 knock-in mouse model was used in order to exclude FOXP3+ natural Treg (nTreg) and depleted the CD4+ T cells isolated from these animals of GFP/FoxP3+ cells.

### Methods

Mice:

C57BL/6 GFP/FoxP3 knock-in mice were used in this Example. CD4+CD25+ and CD4+CD25− T cell populations were isolated using a CD4+ T Regulatory Cell Isolation kit (Miltenyi Biotec) as per the manufacturer's instructions. CD4+FoxP3/GFP+ and CD4+FoxP3+/GFP− T cells were isolated by pre-selection with a Dynal CD4+ T cell negative isolation kit (Invitrogen) and then sorted into both FoxP3/GFP+ and FoxP3/GFP− fractions using a FACS-Vantage Flow Cytometer Cell sorter.

For the in vivo Treg induction, 3×10⁶ CD4+GFP/FoXP3− CD45.2+ cells (autologous donor cells) were embedded into a 300 μl HA/HS/COL gel (Extracel-HP™) along with streptavidin, biotinylated anti-CD3 and anti-CD28 antibodies, and 320 IU/ml IL-2 prior to polymerization with the cross-linked component of the hydrogel mix (PEGSSDA) 30 minutes prior to IP injection. An analogous hydrogel preparation where an equivalent volume of collagen was substituted for the HA/HS component (collagen-only hydrogel) was used as a control.

Four days after IP injection, mice were sacrificed and tissues were harvested. Dissolution of the remaining hydrogel material was achieved per the manufacturer's instructions in order to retrieve cells for analysis. Cells were stained for CD3, CD4 and CD45.2 to allow for discrimination between cells of donor and recipient origins. CD45.1 and CD45.2 are allelic markers that allow one to discern the origin of cells within a mixed population. CD45.1 mice were used as recipients and received either the HA-based hydrogel or the collagen-only hydrogel as an injection into the peritoneal space. On day 4 after implantation, tissues were harvested and stained for intracellular IL-10. Cells were stained and gating was performed to distinguish the CD4+CD3+CD45.2+ (donor cell) and CD4+CD3+CD45.2− (CD45.1+recipient cell) populations.

### Results

FIG. 4A shows the intracellular IL-10 staining of donor cells (CD45.2) and recipient cells (CD45.1) harvested four days after the CD45.2 donor cells had been previously embedded in an immunomodulatory hydrogel and injected into the recipient mouse. As shown in FIG. 4A, enhanced production of IL-10 was observed in the CD45.2+ cells (donor cells) which had been previously embedded in the hydrogel, but not in the CD45.2− (CD45.1 recipient cells). Four days after injection of the hydrogel, a substantial gel volume remained intact within the animals which had received the HA-hydrogel but not the collagen-only hydrogel. As shown in FIG. 4B, after 4 days, the cells remaining embedded in the hydrogel were overwhelmingly CD45.2 positive and expressed IL-10 at a high level.

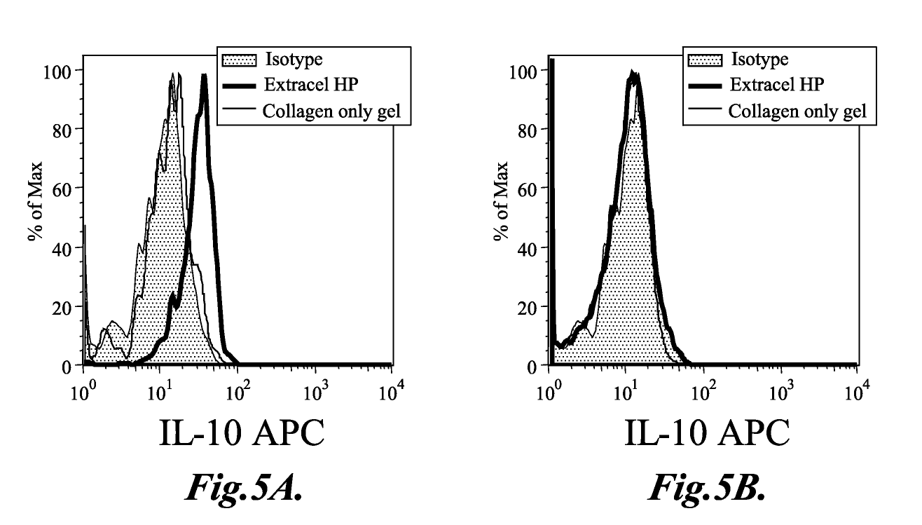

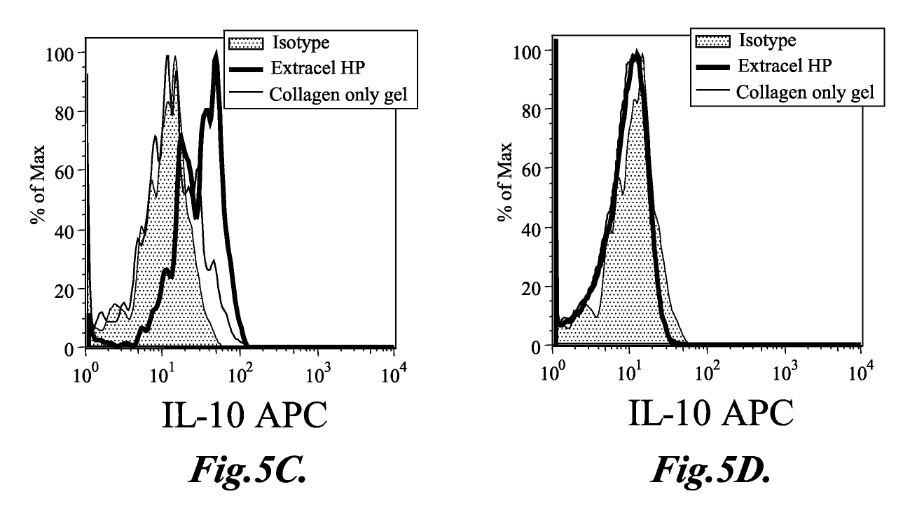

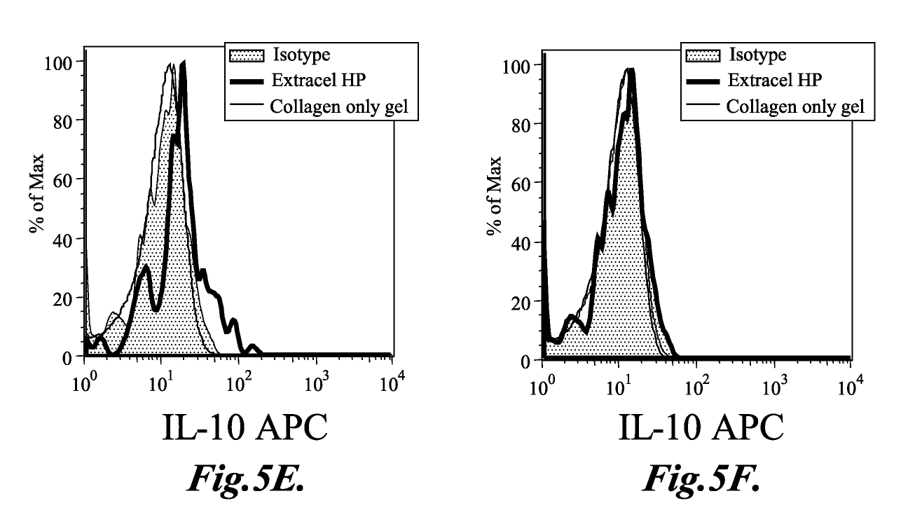

FIG. 5 graphically illustrates IL-10 intracellular staining in cells harvested from the spleen (A,B), mesenteric lymph nodes (C,D) and pancreatic lymph nodes (E,F) 4 days after injection of the CD45.2 donor cells that had been previously embedded in an immunomodulatory hydrogel and injected into the recipient mouse. As shown in FIG. 5, after a period of 4 days, IL-10 producing cells derived from the immunomodulatory hydrogel injection were found in circulation in multiple tissue sites including (1) the spleen (see FIG. 5A CD45.2 donor cells) showing higher IL-2 staining as compared to FIG. 5B (CD45.1 recipient cells); (2) mesenteric lymph nodes (LN), (see FIG. 5C CD45.2 donor cells) showing higher IL-2 staining as compared to FIG. 5D (CD45.1 recipient cells), and (3) pancreatic lymph nodes (LN), (see FIG. 5E CD45.2 donor cells) showing higher IL-2 staining as compared to FIG. 5F (CD45.1 recipient cells). These data are representative of 3 independent experiments.

These results indicate that T cells embedded in a HA-hydrogel express IL-10 in vivo and traffic from the hydrogels into different locations.

### Example 6

This example demonstrates that an HA-based hydrogel containing embedded T cells promotes induction of FoxP3+ regulatory T cells in vivo.

### Rationale

The following experiment was carried out to ascertain whether an HA-based immunomodulatory hydrogel could induce FoxP3 expression in previously FoxP3 negative T cells, and thereby convert them into FoxP3 positive regulatory cells in vivo.

### Methods

Mice: 3×10⁶ CD4+GFP/FOXP3− CD45.2+ cells (donor cells), obtained as described in Example 5, were embedded into a hydrogel preparation HA/HS/COL gel (Extracel-HP™) along with streptavidin, biotinylated anti-CD3 and anti-CD28 antibodies, and 320 IU/ml IL-2, and TGF-beta (50 ng/ml), prior to polymerization with the cross-linked component of the hydrogel mix (PEGSSDA) 30 minutes prior to IP injection.

CD45.1 mice were used as recipients and received the HA/HS/COL hydrogel with embedded T cells as an injection into the peritoneal space. CD45.1 and CD45.2 are allelic markers which allow one to discern the origin of cells within a mixed population. This difference of alleles allowed us to track cells of donor and recipient origin and thereby ascertain the efficiency of in vivo FoxP3 induction using the hydrogel. Moreover, only the CD45.2 animal carried the GFP/FoxP3 allele and all GFP/FoxP3+ cells were depleted prior to use in the experiment, as described in Example 5, therefore any FoxP3+ T cells observed had to have been induced in the hydrogel.

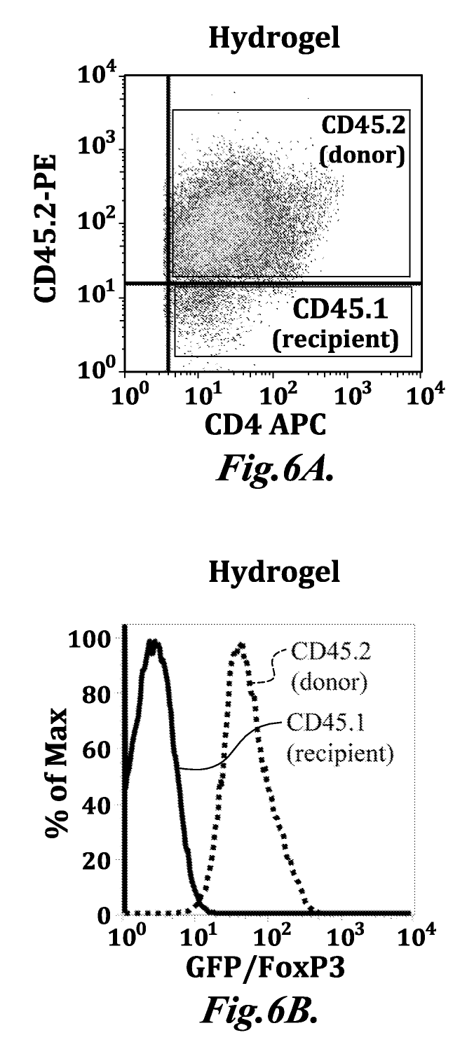

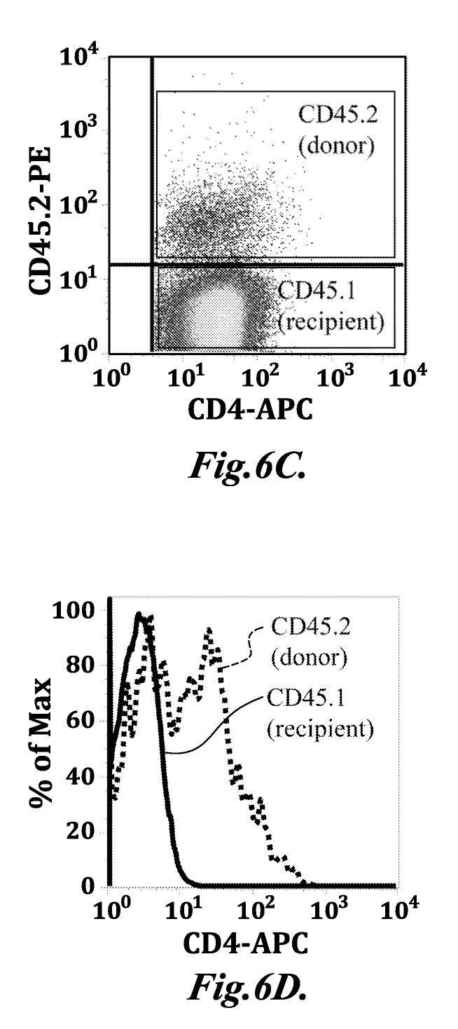

On day 4 after implantation, tissues were harvested and GFP/FoxP3 expression was assessed. A substantial gel volume remained intact within the animals that had received the HA-hydrogel. Cells harvested from the remaining hydrogel in the recipient animal's peritoneum were stained and gated for CD4 and CD45.2 to distinguish the CD4+CD45.2+ (donor cell) and CD4+CD45.2− (recipient cell) populations (FIG. 6).

### Results

FIG. 6A shows the results of FACS analysis of the population of cells harvested from the remaining hydrogel 4 days after implantation, gated for CD4 and stained for CD45.2, showing the CD4+CD45.2+ (donor cells) and CD4+CD45.1− (recipient cells), FIG. 6B shows the results of FACS analysis of the population of cells harvested from the remaining hydrogel 4 days after implantation, gated for CD4 and stained for CD4+CD45.2+ (donor cells) and GFP/FoxP3. As shown in FIG. 6B, the CD45.2− donor cells harvested from the remaining hydrogel were found to express GFP/FoxP3 at a high level.

FIG. 6C shows the results of FACS analysis of the population of cells harvested from the spleen of the recipient animal 4 days after implantation of the hydrogel, gated for CD4 and stained for CD45.2, showing the CD4+CD45.2+ (donor cells) and CD4+CD45.1− (recipient cells). FIG. 6D shows the results of FACS analysis of the population of cells harvested from the spleen of the recipient animal 4 days after implantation of the hydrogel, gated for CD4 and stained for CD4+CD45.2+ (donor cells) and GFP/FoxP3. As shown in FIG. 6C, CD45.2+(donor) cells were found in the spleen at day 4 after transplantation. As shown in FIG. 6D, the CD45.2+donor cells harvested from the spleen were found to express GFP/FoxP3 at a high level.

These results demonstrate that HA-hydrogels containing embedded FoxP3-T cells can induce FoxP3 expression in these embedded T cells and thereby convert them to FoxP3+ regulatory cells in vivo. These results further demonstrate that the T cells induced to express FoxP3+ were found both in the remaining hydrogel as well as the spleen, indicating that they had trafficked out of the hydrogel upon its degradation.

### Example 7

This example demonstrates that GFP/FoxP3+ regulatory T cells induced in vivo by the HA-hydrogel are functional and prevent destruction of allogeneic transplanted tissue.

### Rationale

Type 1 diabetes results from an autoimmune-mediated loss of insulin secreting pancreatic beta cells. Implantation of insulin producing islets has not been successful to date, due in part to re-occurring autoimmunity and insufficient survival of islets.

In order to ascertain whether the GFP/Foxp3+ regulatory T cells induced in vivo, as described in Example 5, are functional, their capacity was tested to forestall an allogeneic tissue transplantation reaction.

### Methods

Pancreatic islets from the mouse strain B6 were transplanted into a mouse of another strain (BALB/c). Because these are different strains, the immune system of the recipient mice can be expected to destroy the islets from the donor strain in the absence of immune tolerance. All mouse work was done in an AALAAC accredited facility and approved by the Benaroya Research Institute IACUC.

Islet isolation: 12-24 week old mice were anesthetized with Avertin (1% Avertin (2,2,2-Tribromoethanol) in tert-amyl alcohol) at 20 μl/g body weight. Immediately following a cut of the descending aorta, pancreata were injected with 4 ml of 4° C. 0.22 μm-filtered 0.8 mg/ml collagenase P (Roche) dissolved in islet media (RPMI 1640 containing 1.0 g NaHCO₃, 10% FBS (Atlanta Biologicals), 1 mM Na-pyruvate, 100 μg/ml penicillin, 100 U/ml streptomycin) through the common bile duct using a 30 ga needle. Pancreata were excised and placed in 50 ml conical tubes on ice. When 2-3 pancreata were obtained, 5 ml of 37° C. islet media was added to each pancreas and incubated at 37° C. for 13 minutes. Warm media was decanted and 30 ml of 4° C. islet media was added to each pancreas. Tubes were shaken vigorously for 1 minute to disrupt the pancreas and then filtered through a 30 mesh metal screen (0.06″ diameter wire). Digest was spun at 4° C. for 10′ at 500 rpm (Beckman GS-6), supernatant decanted off and pellet resuspended in 5 ml 4° C. islet media. 5 ml of 4° C. Histopaque® 1077 (Sigma-Aldrich) was underlayed and the tube spun for 20′ at 2000 rpm (no brake). Liquid above the pellet (islets at the Histopaque®/media interface, 10 ml total) was collected and washed with 40 ml 4° C. islet media for 10′ at 500 rpm. Purified islets were resuspended in 4 ml islet media and placed in 60 mm plates and put in a 37° C. 5% CO₂ incubator. Once all pancreata were processed, islets were picked and counted into a new 60 mm plate using a 200 μl pipette. Islets were cultured overnight and picked again the next day before being placed in the implant. Between 100-150 islet were obtained per B6 mouse.

Implantation of B6 Islets:

The B6 islets were first implanted in polyvinyl acetate (PVA) beads, which were then surgically implanted into the omentum of the BALB/c mouse. The PVA bead was used so that the islets could be retrieved and analyzed for histological evidence of islet survival or destruction. The islet/bead compositions were transplanted in the presence or absence of an HA-hydrogel containing embedded T cells. The HA-hydrogel was generated by embedding 3×10⁶ CD4+GFP/FOXP3− CD45.2+ cells (donor cells), obtained as described in Example 5, into a hydrogel preparation HA/HS/COL gel (Extracel-HP™) along with streptavidin, biotinylated anti-CD3 antibody and 320 IU/ml IL-2 prior to polymerization with the cross-linked component of the hydrogel mix (PEGSSDA) 30 minutes prior to intra-peritoneal (IP) injection.

On the day of surgery, mice were administered Buprenorphine (analgesic 0.05-0.1 mg/kg) prior to the surgery done under isoflurane (anesthesia). Through an approximate 8 mm vertical mid-line incision in the peritoneum, a loop of the small intestine was extracted and the islet implant placed in the intestinal mesentery. The intestinal loop was folded over the implant and placed back into the cavity. Wound closure was done using absorbable sutures (peritoneum) and staples (skin).

Ten days after transplant, the islet/PVA bead implants were retrieved. Removal of islet implants was done in the same manner as implantation. The islets within the beads had no blood supply, but allogeneic tissue would still be expected to initiate a vigorous immune response in the recipient mouse.

Histology:

Implants or pancreata were formalin-fixed and paraffin embedded. Primary antibodies used for antigen detection were: anti-insulin (guinea-pig anti-human, Abcam), anti-Von Willebrand factor (rabbit polyclonal, Abcam). Secondary antibodies used were: fluorescently labeled anti-guinea pig Alexa Fluor® 488 (Molecular Probes IgG (H&L)), goat anti-rabbit (Molecule Probes).

### Results

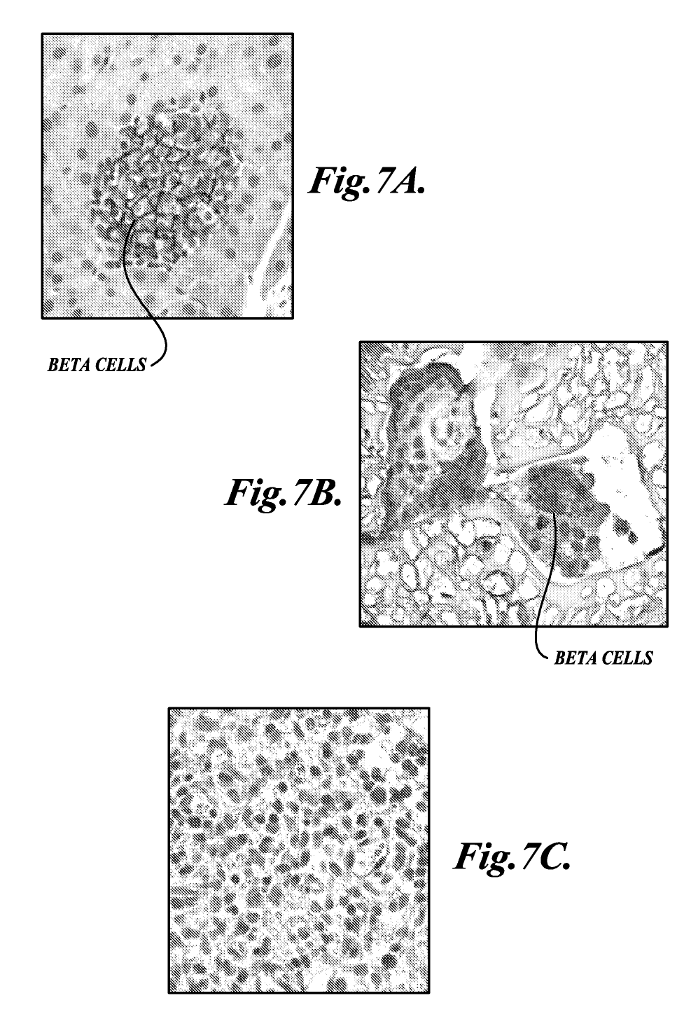

FIG. 7A shows the histological appearance of an islet within a pancreas, with the insulin-producing Beta cells stained darkly for the marker glucagon (see arrow pointing to Beta cells). FIG. 7B is an image of a histological section stained for the marker glucagon (see arrow pointing to Beta cells) taken from a transplanted islet from an animal 10 days after receiving a FoxP3 inducing hydrogel together with an islet/bead construct. FIG. 7C is an image of a histological section stained for the marker glucagon taken from a transplanted islet from an animal 10 days after receiving the islet/bead construct alone.

As shown in FIG. 7B, healthy islets were observed 10 days after transplant in animals that received both the islets and the Fox-P3 inducing hydrogels. Moreover, the PVA bead had undergone substantially less destruction and infiltration with fibrous tissue. In contrast, as shown in FIG. 7C, the animal that received the islets/PVA bead construct alone (without a hydrogel to induce immune tolerance) had no discernable islets at day 10 and had undergone substantially more remodeling infiltration with fibrous tissue such that the PVA and islets had both been obliterated.

These data indicate that the injection of tolerizing hydrogels capable of inducing FoxP3+ Treg are capable of abrogating an immune response against allogeneic tissue.

### Example 8

This example demonstrates that a composition comprising crosslinked HA/HS and IL-10, referred to as “HH-10” provides slow release of IL-10 over a 14 day period in cell culture.

### Rationale

IL-10 is an anti-inflammatory cytokine that promotes immune tolerance in airway inflammation (see, e.g., Vissers, J. L., et al., J. Allergy Clin. Immunol. 113:1204-1210, 2004; Wang, L. H., et al., Int. Arch. Allergy Immunol. 148:199-210, 2009; Matsumoto, K. H., et al., Respirology 14:187-194, 2009; Provoost, S. T., et al., Allergy 64:1539-1546, 2009; Xu, Y. Q., et al., J. Asthma 47:367-373, 2010; Wu, K. Y., et al., Cell. Mol. Immunol. 4:269-275, 2007). A major source of IL-10 in vivo is IL-10 producing Type 1 regulatory T cells (TR1). IL-10 producing TR1 inhibit allergen-specific effector cells and promote immune tolerance in airway inflammation and colitis. The immunosuppressive effects of IL-10, and thereby the TR1 cells producing IL-10, are mediated in large part via suppression of macrophage/monocyte functions, including expression of Class II MHC and co-stimulatory molecules such as IL-12 and CD80/CD86 (Moore, K. W., et al., Annu. Rev. Immunol. 19:683-765, 2001; Roncarolo, M. G., et al., Immunol. Rev. 212:28-50, 2006). TR1 cells are antigen-specific, but once activated, these cells release IL-10 that can mediate bystander suppression against other antigens.

Although desensitization protocols exist for specific allergens, these protocols are arduous and inefficient given that may individuals have multiple allergic triggers. Consequently, there is a need for therapeutic agents for use in treating airway inflammatory disease, such as therapeutic agents capable of inducing immune tolerance.

Adoptive transfer of polyclonal TR1 cells has been shown to ameliorate autoimmunity and allergy in several animal models (Battaglia, M., et al., Diabetes 55:1571-1580, 2006; Groux, H., et al., Diabetes 55:1571-1580, 2006). However, to date, such protocols involving adoptive transfer of polyclonal TR1 cells have shown unacceptable toxicities and the risk of potential exacerbation (Vissers, J. L., et al., J. Allergy Clin. Immunol. 113:1204-1210, 2004; Ahangarani, R. R., et al., J. Immunol. 183:8232-8243, 2009; Fu, C. L., et al., J. Gene Med. 8:1393-1399, 2006). Therefore, the capability to induce autologous regulatory TR1 cells in vivo would be a major advance towards the prevention and treatment of an airway inflammatory disease or condition of the lungs, such as asthma, allergy, chronic obstructive pulmonary disease (COPD), emphysema, autoimmune lung disease, smoke inhalation, acute respiratory distress syndrome (ARDS), cystic fibrosis, laryngitis, bronchitis, pharyngitis, tracheitis, phenumonia, sinusitis, and autoimmune diseases of the lungs.

IL-10 has been used to promote immune tolerance in multiple models (Awasthi, A., et al., Nat Immunol 8:1380-1389, 2007). IL-10 has a short half-life in vivo, limiting its clinical utility as an anti-inflammatory therapeutic agent (Rachmawati, H., et al., J. Pharm. Sci. Technol. 65:116-130, 2011; Fuchs, A., et al., J. Clin. Immunol. 16: 291-303, 1996; Li, L., et al., J. Immunol. 153:3967-3978, 1994). Numerous methods have been attempted to administer IL-10 in a more long-lasting manner; however, all methods attempted to date are problematic or inefficient (see, e.g., Ahangarani, R. R., et al., J. Immunol. 183:8232-8243, 2009; Contrera, X., et al., Microbes Infect. 6:1182-1190, 2004; Fu, C., et al., Clin. Exp. Immunol. 153:258-268, 2008; Zheng, X., et al., J. Immunol. 158:4507-4513, 1997; Frossard, C., et al., J. Allergy Clin. Immunol. 119:952-959, 2007; Houri-Haddad, Y., et al., J. Dent. Res. 86:560-564, 2007; Bhaysar, M., et al., Gene Ther. 15:1200-1209, 2008; Buff, S., et al., Gene Ther. 17:567-576, 2010).

In tissues, numerous cytokines and growth factors are bound to heparin or the closely related molecule heparan sulfate (HS), possibly prolonging their half-life (Gabr, A., et al., Respir. Res. 8:36, 2007; Wang, Z. et al., Zhongguo Wei Zhong Bing Ji Jui Yi Xue 23:239-242, 2011). Heparin is a sulfated glycosaminoglycan that has the ability to bind charged molecules. Heparan sulfate (HS) is particularly abundant in basement membranes (BM) where it acts as a reservoir for cytokines and growth factors (Tanaka, Y., et al., Proc. Assoc. Am. Physicians 110:118-125, 1998). There is evidence to suggest that T cells and other leukocytes can utilize cytokines bound to heparin or HS (Wrenshall, L., et al., J. Immunol. 163:3793-3800, 1999; Wrenshall, L., et al., J. Immunol. 170:5470-5474, 2003).

In this Example, an experiment was carried out to determine the ability of a composition comprising HA/HS and IL-10 to deliver IL-10 over time in cultured cells.

### Methods

A composition referred to as “HH-10” was prepared which contains the following agents:
(i) high MW hyaluronan (HA) (concentration from 0.02% to 2% w/v),
(ii) heparin (HI), or heparan sulfate (HS) (concentration from 0.02% to 2% w/v); and
(iii) recombinant IL-10 (concentration from 1 pg/ml to 1 mg/ml)

The HH-10 composition was crosslinked in order to promote its stability and longevity. As described herein, the compositions comprising HA, HA/HS and HA/HS and IL-10 can be crosslinked using chemical or non-chemical crosslinking agents. For example, the crosslinking of hyaluronan and heparin or heparan sulfate can be carried out via a non-chemical process, such as radiation treatment (i.e., electron beams, gamma rays, x-rays, ultraviolet light); or via a chemical crosslinking process such as crosslinking with biscarbodiimide, protein crosslinking or internal esterification (HAACP). For example, commercially available cross-linked hyaluronan preparations include Incert (crosslinked with a biscarbodiimide) and Synvisc® or Restylane® (protein cross-linked).

In one embodiment, derivatives providing for covalently crosslinked networks are present. An exemplary crosslinked matrix derived from a water-soluble, UV crosslinkable polymer comprises poly(ethylene glycol) dimethylacrylate (PEGDMA), which is used in conjunction with the conjugation of thiol groups to the glycosaminoglycans in question which are crosslinked by PEGDMA. In this experiment, the chemically modified HA and heparin were used that had thiol groups conjugated to them, and were crosslinked using PEGSSDA.

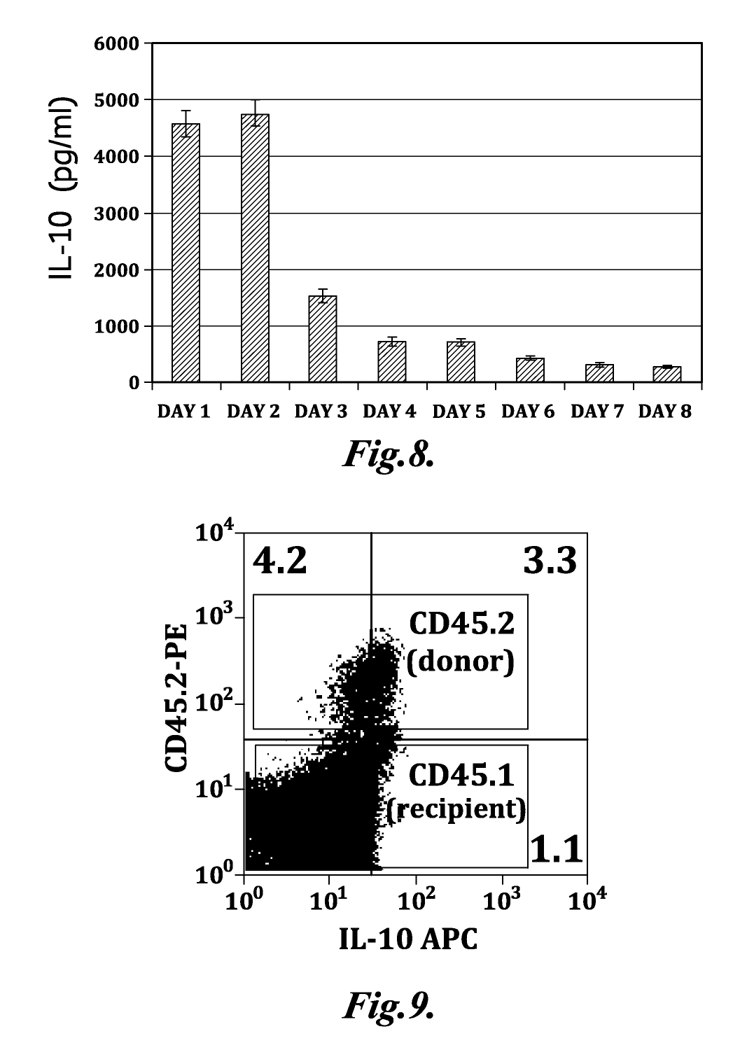

In the embodiment of HH-10 tested in this experiment, a 200 μl volume of the crosslinked HA/HS gel containing 20,000 pg/ml of IL-10 was incubated overnight in 200 μl of RPMI media. The RPMI was then removed after 24 hours and stored for subsequent analysis of IL-10, and replaced with 200 μl of fresh RPMI media. This process was repeated every 24 hours for 8 days. This experiment was performed two times. The results of one representative experiment is shown in FIG. 8.

### Results

FIG. 8 graphically illustrates the slow release of IL-10 from HH-10 (crosslinked HA/HS and IL-10) over time in cell culture media (RPMI). As shown in FIG. 8, for the HA/HS gel containing 20,000 pg/ml of IL-10, the average total IL-10 retained by the gel at the end of 8 days was 13,341 pg, with a standard deviation of ˜6%. This was equivalent to a retention rate of at least 67% of the original IL-10 loaded into the HA/HS gel. It was further determined that even at the end of 14 days, the HA/HS gels were still releasing IL-10 at levels over baseline (data not shown). Accordingly, this Example demonstrates that HH-10 comprising HA/HS and IL-10 slowly releases IL-10 over at least a 14-day period.

As further described in Examples 9 and 10, delivery of IL-10 using a composition containing IL-10 bound to HS, such as HH-10, and delivery as an aerosol is a novel solution to the problem of IL-10 delivery. Low molecular weight HS and Heparin (HI) have been aerosolized for clinical applications (Wang, Q., et al., Jpn. J. Pharmacol. 82:326-330, 2000; Murakami, K., et al., Shock 18:236-241, 2002; Gabr, A., et al., Respir. Res. 8:36, 2007; Wang, Z., et al., Zhongguo Wei Zhong Bing Ji Jiu Xue 23:239-242, 2011; Seeds, E., et al., Pulm. Pharmacol. 8:97-105, 1995). In these experiments, HS or HI is typically solubilized in saline and administered using a standard nebulizer. Low MW HI has been delivered in this manner at concentrations between 100-2000 IU/ml.

### Example 9

This Example demonstrates that crosslinked hyaluronan (high MW HA), heparin and collagen (HA/HS/COL hydrogel) is capable of inducing endogenous TR1 cells to produce IL-10 in vivo when implanted into mice.

### Rationale

As described in Examples 4-6 herein, it has been demonstrated that high MW crosslinked hyaluronan, a component of HH-10, promotes the resolution of inflammation through induction of TR1 cells and production of IL-10 (see also Bollyky, P., et al., Proc. Natl. Acad. Sci. USA 108:7938-7943, 2011, incorporated herein by reference).

Hyaluronan is a long disaccharide with both structural and immunologic functions (Laurent, T., et al., FASEB J. 6:2397-2404, 1992). The size of hyaluronan provides contextual cues to leukocytes regarding the stage of injury and its resolution (Stern, R., et al., Eur. J. Cell. Biol. 85:699-715, 2006; Sorokin, L., Nat. Rev. Immunol. 10:712-723, 2010). Fragmentary hyaluronan (low molecular weight HA) predominates at sites of infection and acute injury (Stern et al., 2006, supra), is a Toll-like receptor (TLR) ligand and is proinflammatory (Jiang, D., et al., Nat. Med. 11:1173-1179, 2005; Powell, J., et al., Immunol. Res. 31:207-218, 2005). In contrast, intact hyaluronan (high molecular weight HA) is found in healing or uninjured tissues (Stern et al., 2006, supra), does not bind TLR, and is anti-inflammatory (Teder, P., et al., Science 296:155-158, 2002; Termeer, C., et al., J. Exp. Med. 195:99-111, 2002; Deed, R., et al., Int. J. Cancer 71:251-256, 1997; Delmage, J., et al., Ann. Clin. Lab. Sci. 16:303-310, 1986).

As demonstrated in the Examples herein, the inventors have found that intact, but not fragmentary hyaluronan promotes induction of IL-10 producing TR1 cells in both murine and human systems. These TR1 cells were capable of abrogating disease in an IL-10 dependent mouse colitis model, as described herein and further demonstrated in Bollyky, P., et al., 2011, supra. The Treg precursor cells in the colitis model were CD4+CD62L-FoxP3−, suggesting that effector memory cells assume a regulatory phenotype when they encounter their cognate antigen in the context of intact (high MW) HA. As described in Bollyky, P., et al., 2011, supra, this Treg induction by intact (high MW) HA was dependent on concomitant low-dose T cell receptor ligation and was potentiated by low-dose IL-2. It was also found that IL-10 production was dependent on MAP kinase signaling through p38 and ERK1/2. These findings are consistent with the results described herein that intact (high MW) HA enhanced the function of FoxP3+ natural regulatory T cells (nTreg) through enhanced production of IL-10 (see also Bollyky, P., et al., J. Immunol. 179:744-747, 2007; Bollyky, P., et al., J. Immunol. 183:2232-2241, 2009). In sum, these studies suggest that intact (high MW) HA crosslinks CD44 and this functions as a tissue integrity cue which promotes induction of IL-10 producing TR1 regulatory T cells (FoxP3−).

In this Example, an experiment was carried out to determine if crosslinked high MW HA/heparin/collagen could generate TR1 cells in vivo when implanted into mice via intraperitoneal or subcutaneous injection.

### Methods

3×10⁶ CD4+GFP/FOXP3-CD62L-CD45.2+ cells (donor cells) were injected into the peritoneal space of CD45.1 recipient mice together with one of the following:
(i) crosslinked hyaluronan (high MW HA), heparin and collagen (HA/HS/COL hydrogel) (Extracel-HP™, Glycosan Biosystems), crosslinked with polyethylene glycol diacrylate (PEGDA); or
(ii) crosslinked collagen only.

Four days after injection, lymph node and spleenocytes from recipient animals were harvested and stained for intracellular IL-10 and analyzed by FACS analysis.

### Results

FIG. 9 shows the results of FACS analysis after gating was performed on cells harvested from mice implanted with crosslinked HA/HS/COL or crosslinked COL control to sort the CD45.2+ (donor) and CD45.2-CD45.1+ (recipient) cells. The gating shown in FIG. 9 was carried out to allow for the discrimination between recipient (CD45.1) and donor (CD45.2) cells. Donor cells (CD45.2) were injected into the peritoneum at the same time as crosslinked HA/HS/COL, and therefore would be expected to come into contact with the HA/HS/COL, in contrast to the recipient spleenocytes and lymph node (CD45.1) cells.

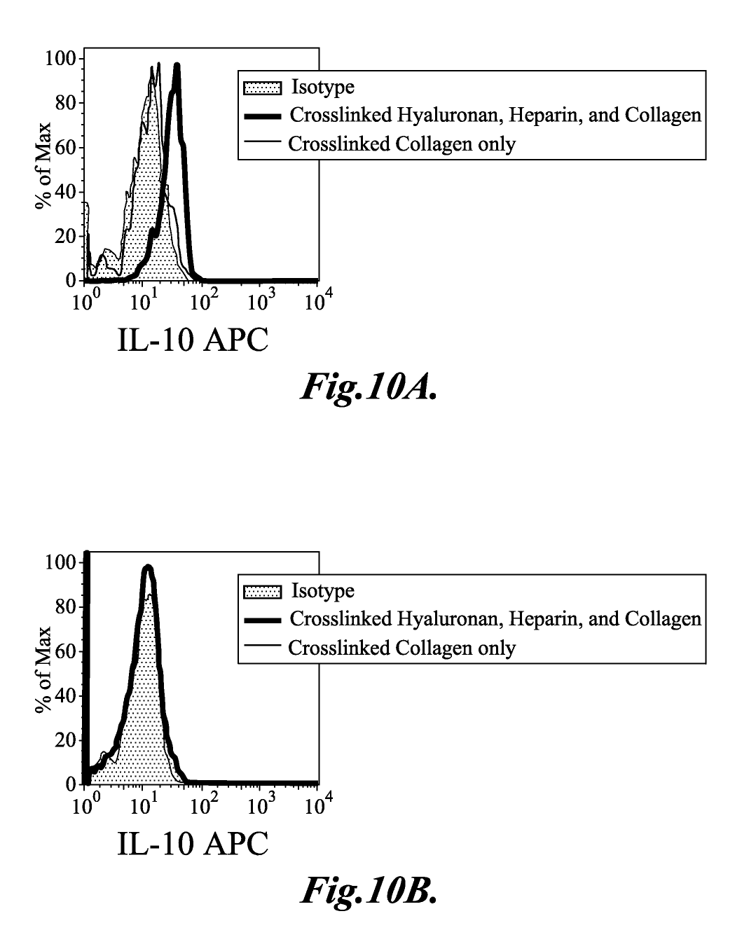

FIG. 10A shows the results of FACS analysis of IL-10 positive spleen donor cells (CD45.2+) obtained from mice that were implanted with (i) the crosslinked HA/HS/Col hydrogel, or (ii) the crosslinked COL control gel in comparison to spleen cells from the isotype control mice.

FIG. 10B shows the results of FACS analysis of IL-10 positive spleen recipient cells (CD45.1+) obtained from mice that were implanted with (i) the crosslinked HA/HS/Col hydrogel, or (ii) the crosslinked COL control gel in comparison to spleen cells from the isotype control mice.

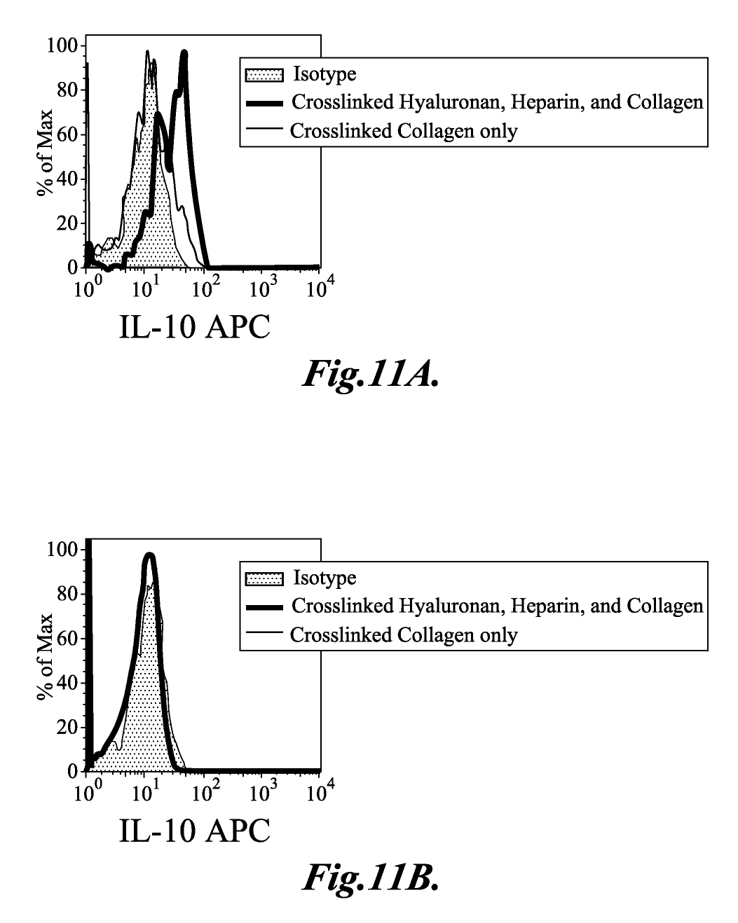

FIG. 11A shows the results of FACS analysis of IL-10 positive mesenteric lymph node (LN) donor cells (CD45.2+) obtained from mice that were implanted with (i) the crosslinked HA/HS/Col hydrogel, or (ii) the crosslinked COL control gel in comparison to spleen cells from the isotype control mice.

FIG. 11B shows the results of FACS analysis of IL-10 positive mesenteric lymph node (LN) recipient cells (CD45.1+) obtained from mice that were implanted with (i) the crosslinked HA/HS/Col hydrogel, or (ii) the crosslinked COL control gel in comparison to spleen cells from the isotype control mice.

The data shown in FIGS. 10 and 11 are representative of three independent experiments, and demonstrate that the implanted hydrogels generated TR1 cells in vivo.

These results demonstrate that crosslinked hyaluronan (high MW HA), heparin and collagen (HA/HS/COL hydrogel) is capable of inducing endogenous TR1 cells to produce IL-10 in vivo when implanted into mice. At sites of inflammation, such as inflamed airways, a composition comprising crosslinked HA/HS and IL-10 (HH-10) would therefore be expected to come into contact with T cells, which would be expected to behave like the CD45.2 cells in this experiment.

These results also indicate that T cells that came into contact with crosslinked HA and heparin were induced to produce IL-10. Furthermore, these cells then trafficked to multiple sites, indicating that they were viable and that their production of IL-10 was phenotypically stable outside of the environment of the crosslinked hyaluronan and heparin.

### Example 10

This Example describes the use of an OVA-sensitization mouse model of airway hypersensitivity (AHS) to adapt the TR1 induction system that has been successfully used for TR1 induction in vitro and in vivo using IP injection, as described in Example 9, for use in airway delivery for treatment of respiratory disease.

### Rationale

As described herein in Examples 4-6 and Example 9, compositions comprising high MW hyaluronan (HA) promote the resolution of inflammation through induction of TR1 cells and production of IL-10 (see also Bollyky, P., et al., Proc. Natl. Acad. Sci. USA 108:7938-7943, 2011, incorporated herein by reference).

One major challenge for use of HA as a therapeutic agent in vivo is maintaining HA in intact, high MW form. Uncrosslinked hyaluronan rapidly breaks down into smaller, proinflammatory fragments (Laurent, T., et al., FASEB J. 6:2397-2404, 1992; Stern, R., et al., 2006, supra). As described herein, TR1 induction signals can be recapitulated using synthetically crosslinked hyaluronan (see also Bollyky, P., et al., 2011, supra).

In this Example, the use of an OVA-sensitization mouse model of airway hypersensitivity (AHS) to adapt the TR1 induction system that has been successfully used for TR1 induction in vitro and in vivo using IP injection, as described in Example 9, for use in airway delivery for treatment of respiratory disease is described.

### Methods

An ovalbumin (OVA) sensitization mouse model of airway hypersensitivity (AHS), as described in (Cheng, G., et al., Matrix Biol. 30:126-134, 2011), is used to evaluate that use of intact HA-based treatments for airway inflammation. In addition to intact HA, Heparan sulfate (HS), a glycosaminoglycan capable of binding certain cytokines and growth factors, such as IL-10, can be added to the solution to deliver IL-10 to drive TR1 induction.

In this OVA-sensitization model, mice are sensitized to OVA in adjuvant and subsequently challenged with OVA aerosols. This system provides the ability to manipulate the ECM at the site of disease in a model that is known to be highly responsive to treatment with nTreg or exogenous IL-10 (Vissers, J., et al., J. Allergy Clin. Immunol. 113:1204-1210, 2004; Burchell, J., et al., Am. J. Physiol. Lung Cell. Mol. Physiol. 296:L307-L319, 2009; Fu, C., et al., J. Gene Med. 8:1393-1399, 2006).

Airway hypersensitivity (AHS) is then assessed using cellular, physiologic and histological assays. These assays include analysis of bronchoalveolar lavage fluid (BALF) for cytokines, cell counts, and eosinophilia; analysis of serum for IgE; physiologic assessments of bronchial hyper-responsiveness to methacholine challenge, and histologic analysis of airway hyper-responsiveness and remodeling. The induction of TR1 in the mice treated with the nebulized compositions is also evaluated.

The following compositions will be delivered as nebulized compositions before, during, or after initial OVA sensitization:

(i) cross-linked, high MW HA;

(ii) cross-linked, high MW HA and HS;

(iii) cross-linked, high MW HA and HS plus IL-10

Negative controls will include: nebulized PBS, non-crosslinked HA and non-crosslinked HS.

The impact of this therapy on AHS upon re-challenge with OVA will also be evaluated.

In some embodiments, intact, high MW HA is solubilized in water or phosphate buffered saline (PBS). The concentration of HA is between 0.001% and 10% (w/v). In some embodiments, intact (high MW) HA is crosslinked to promote its longevity and stability. The HA may be crosslinked prior to dilution in water or PBS, or the solution containing solubilized HA may be crosslinked.

The solution containing high MW crosslinked HA is aerosolized with a nebulizer and delivered to the airway of a mammalian subject for use as a valuable, inexpensive and biocompatible therapy for treatment of allergies and asthma.

In some embodiments, the intact HA and HS are crosslinked, either separately prior to combining, or in a solution comprising both HA and HS suitable for inhalation.

In one embodiment, intact HA, HS and IL-10 are combined in a solution suitable for inhalation at concentrations effective to treat airway inflammation, and the solution is then treated with a crosslinking agent. After crosslinking, the solution is administered to a subject for treatment.

In some embodiments, the intact crosslinked HA is delivered to a subject in conjunction with at least one of Heparan sulfate (HS), Heparin (HI), or collagen (Col) to improve its stability. In some embodiments, the concentration of HI is between 1 and 10,000 IU/ml. In some embodiments, the concentration of HS is between 0.001% and 10% (w/v). In some embodiments, the HS and/or HI are also crosslinked (either separately, or in a solution in combination with HA) in order to promote the stability and longevity of the solution. In some embodiments, HS or HI is used to deliver recombinant IL-10. In some embodiments, the concentration of HI is between 1 and 10,000 IU/ml. In some embodiments, the concentration of HS is between 0.001% and 10% (w/v). In some embodiments, the HS or HI is crosslinked. In some embodiments, the HS or HI is delivered via aerosolization in conjunction with hyaluronan (HA) or collagen. In such embodiments, the HA or collagen are included at a concentration of between 0.001% and 10% (w/v), and can be crosslinked.

Based on the results described herein in Examples 8 and 9, the nebulized crosslinked intact HA/HS/IL-10 composition is expected to be effective to induce TR1 in vivo, and the induced TR1 are expected to effectively treat AHS in this model.

### Example 11

### Therapeutic Compositions and Methods for Treating Airway Inflammatory Diseases

### Rationale

In asthmatic patients, oral or inhaled steroids are often used to suppress the immune response. Steroids engender a generalized immune suppression that can lead to infection and a variety of other complications. Allergan-induced asthma is caused by a loss of immune tolerance to environmental aeroallergens. This tolerance is enforced by populations of regulatory T-cells. The best studied of these regulatory T cells are FoxP3+ Treg and TR1 cells. Both of these regulatory T cell subsets mediate immune tolerance to self and foreign antigens via production of prodigious amounts of IL-10. The immunosuppressive effects of IL-10 are mediated in large part via suppression of macrophage/monocyte functions, including expression of class II MHC and co-stimulatory molecules such as IL-2 and CD80/CD86. Disorders in IL-10 production of signaling are implicated in asthma and allergy.

Because asthma is a disease of immune dysregulation, one appealing strategy for treatment of asthma is to bolster the body's natural mechanisms for immune regulation, including regulatory T cells and the immunoregulatory cytokine IL-10. There is evidence that these regulatory mechanisms are impaired in asthma. The number of regulatory T cells are often low in asthma, and their transfer in animal models has shown benefit. However, protocols carried out in prior studies for inducing either TR1 or FoxP3+ Treg in situ come with unacceptable toxicities and the risk of potential exacerbation. Moreover, attempts to provide exogenous IL-10 even through inhalation are limited by the short half life of this molecule in vivo.

As described in the examples herein, a role for intact hyaluronan (HA) in the induction and function of regulatory T cells is identified. HA is a component of the extracellular matrix (ECM) of healing tissues whose size tracks with the stage of inflammation. Intact, high-molecular weight HA (HMW-HA) predominates in non-inflamed or healing tissues and is anti-inflammatory, while fragmentary low-molecular weight HA predominates in inflamed tissues and is pro-inflammatory. Intact but not fragmentary HA binds to CD44 and promotes the persistence and function of FoxP3+ Treg. Our data suggests that in this context, HMW-HA functions as a tissue integrity cue that promotes the induction of IL-10 producing TR1 from memory T-cell precursors. This suggests that when memory Tregs encounter their cognate antigen in the context of high-molecular weight HA, they adopt a regulatory phenotype.

A composition comprising cross-linked high molecular weight (intact) hyaluronan (XHA) and at least one allergen, such as an aeroallergen, that is capable of promoting the induction of immune tolerance to the allergens that trigger asthma in a mammalian subject is described herein. The composition comprising XHA and at least one allergen prevent the inflammatory response to allergens, whereas the currently available therapies merely suppress the symptoms of that response. Therefore, the inventive compositions and methods described herein vastly improve the therapeutic options for asthmatics.

### Methods and Results

1. XHA Promotes TR1 Induction

Methods:

Cross-linked high molecular weight hyaluronan (XHA) was generated by using thiol-labeled hyaluronan and a polyethylene glycol as a cross-linker.

CD4+CD62L-GFP/FoxP3− memory T cells were activated with anti-CD3/CD28 antibodies and IL-2 for 96 hours in the presence or absence of 1% XHA. The cells were analyzed for intracellular IL-10 staining by FACS analysis, and the concentrations of various cytokines including IL-10 under the same conditions were analyzed in the culture supernatants.

Results:

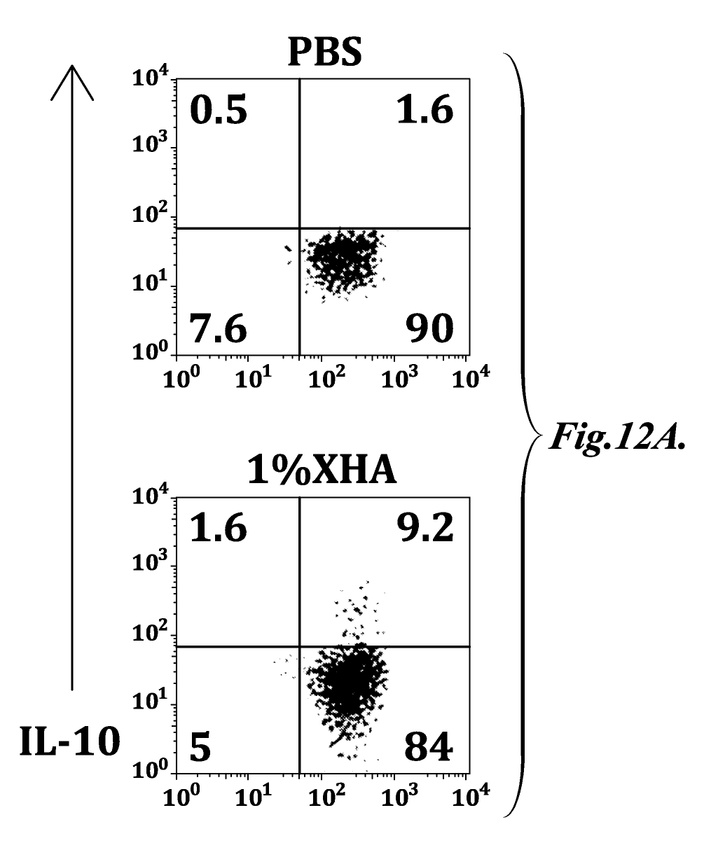

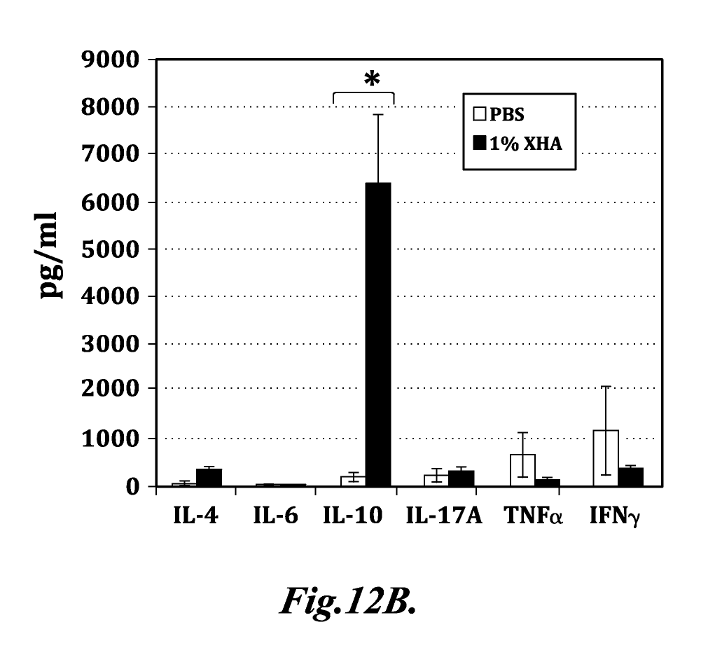

FIG. 12A graphically illustrates the results of FACS analysis analyzing intracellular IL-10 staining of CD4+CD62L-GFP/FoxP3− memory T cells from mice that were activated with anti-CD3/CD28 antibodies and IL-2 for 96 hours in the absence (top panel, PBS control) or in the presence of 1% XHA (bottom panel).

FIG. 12B graphically illustrates the concentrations of cytokines IL-4, IL-6, IL-10, IL-17A, TNFα and IFNγ in CD4+CD62L-GFP/FoxP3− memory T cells from mice that were activated with anti-CD3/CD28 antibodies and IL-2 for 96 hours in the absence (PBS control) or presence of 1% XHA (N=4 experiments, symbol “*” indicates statistical significance).

As shown in FIGS. 12A and 12B, XHA induced IL-10 production by memory T cells. The XHA-mediated induction of IL-10 was specific, in that other cytokines (I-4, IL-6, IL-17A, TNF-alpha and IFN-gamma) were not significantly increased (FIG. 12B). This effect was dependent on concomitant TCR ligation and on cross-linking of CD44, the HA receptor. XHA-induced IL-10 production was generated from both mouse and human memory T cell precursors and these had regulatory function, establishing that these cells function as TR1. No IL-10 production was seen in the setting of either Matrigel® or fibrin gel controls (data not shown). Matrigel® and fibrin gels were included in the experiment as negative controls, and demonstrate the specificity of HA, and that not every matrix material promotes IL-10 induction.

These results demonstrate that XHA is able to induce IL-10 producing TR1 regulatory T cells from memory cells, indicating that XHA is an ideal therapeutic for induction of T cell tolerance.

2. Intranasal Administration of XHA Promotes TR1 Induction and Ameliorates Airway Hypersensitivity

Methods: A well-established OVA mouse model of airway hypersensitivity (Zhou et al., Nat Immunol 10:1047-53, 2005) was used to evaluate the pro-tolerogenic attributes of XHA. In an experiment carried out using substantially similar methods to the methods described in Example 10, it was determined whether XHA would function in vivo to suppress an airway inflammatory response while inducing IL-10 producing T cells.

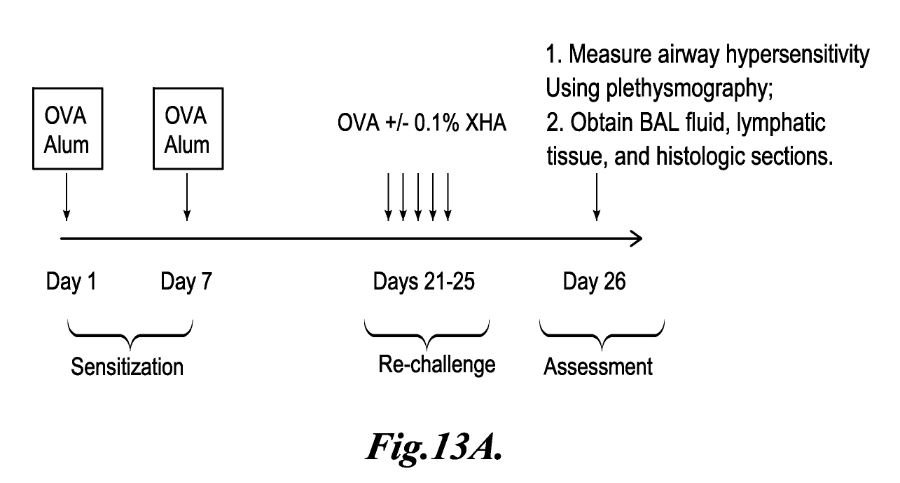

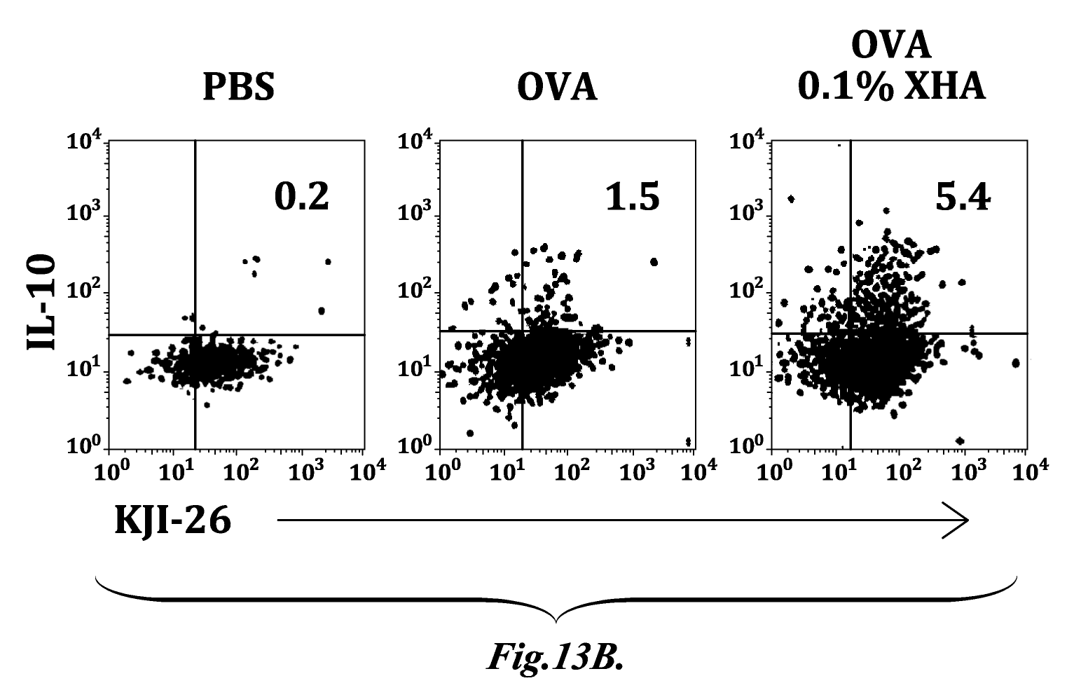

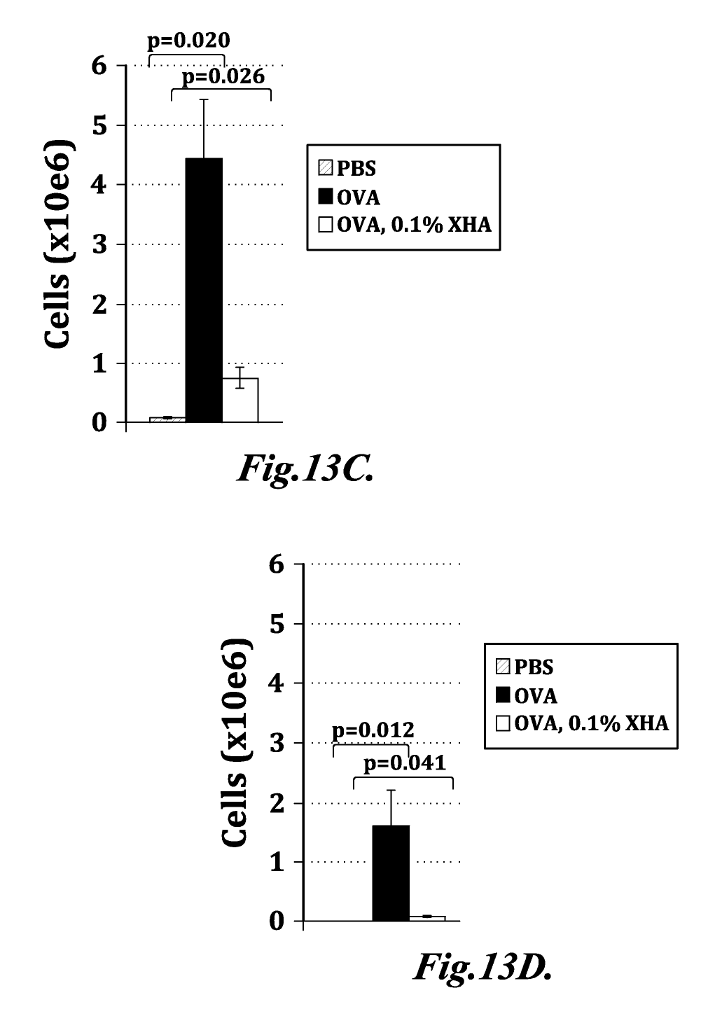

As illustrated in FIG. 13A, in this model, D0.11 Rag−/−mice carrying a TCR specific for OVA were sensitized to ovalbumin (OVA), a chick egg protein, and the adjuvant Alum on days 1 and 7. Starting on day 21, animals were re-challenged intra-nasally with either PBS, OVA or OVA+0.1% XHA for five days. On day 26, bronchoalveolar lavage (BAL) fluid was removed and evaluated for IL-10 producing CD4+OVA-specific T cells, total cells and eosinophilia.

Results:

FIG. 13A illustrates the time line of events in the mouse model of airway hypersensitivity wherein ovalbumin (OVA) a chick egg protein, serves as an antigenic trigger, used to demonstrate that XHA ameliorates airway hypersensitivity in a mouse model.

FIG. 13B graphically illustrates FACS analysis of lymphocytes, gated for CD4+ T-cells, that were isolated at day 26 from D0.11 Rag−/−mice carrying a TCR specific for OVA that were treated with PBS control, OVA alone, or OVA+0.1% XHA according to the protocol shown in FIG. 13A, stained for intracellular IL-10 and KJI-26, an antibody clone that recognizes the DO.11 Ova-specific TCR (data is representative of two experiments). In DO11.10 Rag−/−mice, any cell that is positive for the DO11.10 TCR by definition specifically recognizes OVA as its cognate antigen. Therefore, staining for the DO11.10 TCR using the KJI-26 allows one to track OVA-specific T-cell responses. This model was used to demonstrate that IL-10+ TR1 cells induced using XHA specifically recognize OVA.

As shown in FIG. 13B, intranasal administration of 0.1% of XHA resulted in an increase of IL-10 producing CD4+KJI-260va-specific T cells. Together with the work with Balb/c mice, a non-antigen-specific model, these two pieces of data demonstrate that XHA delivered with OVA can suppress OVA-induced airway hyper-sensitivity and that XHA delivered with OVA can induce IL-10 producing T-cells that are specific to OVA.

FIG. 13C graphically illustrates the total cells in bronchoalveolar lavage (BAL) fluid obtained at Day 26 from conventional Balb/C mice that were treated with PBS control, OVA alone, or OVA+0.1% XHA according to the protocol shown in FIG. 13A.

FIG. 13D graphically illustrates the number of eosinophils in bronchoalveolar lavage (BAL) fluid obtained at Day 26 from conventional Balb/C mice that were treated with PBS control, OVA alone, or OVA+0.1% XHA according to the protocol shown in FIG. 13A.

As shown in FIGS. 13C and 13D, mice administered with XHA demonstrated a decrease in both the total number of cells and number of eosinophils in BAL fluid. These data validate that XHA ameliorates airway hypersensitivity in a mouse model. As noted above, Rag−/− mice were used in FIG. 13B because it was the only way to look at IL-10 production in OVA-specific (antigen specific) cells. For measuring cell number in fluid, any mice can be used for this (e.g. Balb/C mice).

In summary, the results shown in this Example demonstrate that airway treatment with XHA promotes antigen-specific TR1 induction in vivo and ameliorates disease in a mouse asthma model. As demonstrated in this Example, inhaled XHA creates a pro-tolerogenic environment that builds immune tolerance to ambient allergens. This unique approach to inducing TR1 in a polyclonal manner, has significant potential to augment the underlying mechanisms of asthma, and other airway inflammatory diseases.

While illustrative embodiments have been illustrated and described, it will be appreciated that various changes can be made therein without departing from the spirit and scope of the invention.

1. A composition for treating an airway inflammatory disease comprising (i) high molecular weight, crosslinked hyaluronan, and (ii) at least one aeroallergen.

2. The composition of claim 1, wherein the composition is formulated as an inhalable solution or an intranasal solution.

3. The composition of claim 2, wherein the inhalable solution comprises water or saline solution.

4. The composition of claim 2, wherein the inhalable solution or intranasal solution comprises hyaluronan at a concentration of from 0.001% to 10% (w/v).

5. The composition of claim 1, wherein the aeroallergen is selected from the group consisting of pollen, spores, mold, animal dander, and dust mites.

6. The composition of claim 3, wherein the high molecular weight hyaluronan and at least one aeroallergen are subjected to a crosslinking agent after dilution in the saline solution.

7. A method of inducing immune tolerance to one or more aeroallergens in a mammalian subject suffering from or at risk for developing an airway inflammatory disease, the method comprising:
administering a composition comprising high molecular weight, crosslinked hyaluronan to a mammalian subject in an amount effective to induce immune tolerance to one or more aeroallergens in the mammalian subject.

8. The method of claim 7, wherein the composition further comprises at least one aeroallergen.

9. The method of claim 7, wherein the composition is formulated as an inhalable solution comprising water or saline solution which is administered to an airway of the subject.

10. The method of claim 7, wherein the composition is formulated as an intranasal solution which is administered to a nasal passageway of the subject.

11. The method of claim 9, wherein the inhalable solution is administered to the subject with a nebulizer.

12. The method of claim 9, wherein the inhalable solution comprises hyaluronan at a concentration of from 0.001% to 10% (w/v).

13. The method of claim 8, wherein the aeroallergen is selected from the group consisting of pollen, spores, mold, animal dander, and dust mites.

14. The method of claim 7, wherein the mammalian subject is a human subject.

15. The method of claim 14, wherein the human subject is suffering from or at risk for developing an airway inflammatory disease or condition of the lungs selected from the group consisting of: asthma, allergy, chronic obstructive pulmonary disease (COPD), emphysema, autoimmune lung disease, smoke inhalation, acute respiratory distress syndrome (ARDS), cystic fibrosis, laryngitis, bronchitis, pharyngitis, tracheitis, pneumonia, and sinusitis.

16. A method for treating a human subject suffering from an airway inflammatory disease comprising administering to the subject a composition comprising high molecular weight, crosslinked hyaluronan in an amount effective to induce immune tolerance to at least one aeroallergen in the human subject.

17. The method of claim 16, wherein the composition further comprises at least one aeroallergen.

18. The method of claim 16, wherein the composition is formulated as an inhalable solution comprising water or saline solution which is administered to an airway of the subject.

19. The method of claim 16, wherein the composition is formulated as an intranasal solution which is administered to a nasal passageway of the subject.

20. The method of claim 18, wherein the inhalable solution is administered to the subject with a nebulizer.

21. The method of claim 18, wherein the inhalable solution comprises hyaluronan at a concentration of from 0.001% to 10% (w/v).

22. The method of claim 16, wherein the human subject is suffering from or at risk for developing an airway inflammatory disease or condition of the lungs selected from the group consisting of: asthma, allergy, chronic obstructive pulmonary disease (COPD), emphysema, autoimmune lung disease, smoke inhalation, acute respiratory distress syndrome (ARDS), cystic fibrosis, laryngitis, bronchitis, pharyngitis, tracheitis, pneumonia, and sinusitis.
